(19) United States
(12) Patent Application Publication
(10) Pub. No.: US 2026/0216178 A1
(43) Pub. Date: Jul. 30, 2026

(54) TREATMENT OF PSYCHIATRIC DISORDER

(71) Applicant: GSKB Pharmaceuticals, LLC, Naperville, IL (US)

(72) Inventors: Sandeep GAONKAR, Naperville, IL (US); Kara MULLIGAN, Naperville, IL (US); Paul Sudhakar, Shawnee, KS (US); Scott BOYER, West Chicago, IL (US)

(21) Appl. No.: 19/461,632

(22) Filed: Jan. 27, 2026

Related U.S. Application Data
(60) Provisional application No. 63/750,055, filed on Jan. 27, 2025.

(51) Int. Cl. A61K 31/496; A61K 9/00; A61K 31/517

(52) U.S. Cl. CPC A61K 31/496; A61K 9/0053; A61K 31/517

(57) ABSTRACT

The present invention provides for a method of treating a person suffering from a psychiatric disorder that includes administration of a pharmaceutically effective amount of (i) nefazodone, or a pharmaceutically acceptable salt thereof, and (ii) risperidone; in a specified dosing regimen. Also provided is a unit dose that includes (i) one or more pharmaceutically acceptable excipients, and a specified amount of (ii) nefazodone, or a pharmaceutically acceptable salt thereof, and (iii) risperidone. Also provided is a therapeutic package that includes (a) the unit doses, (b) a finished pharmaceutical container that contains the unit doses, (c) printed indicia, and (d) optionally an outside container containing the finished pharmaceutical container and the printed indicia.

### CROSS-REFERENCE TO RELATED APPLICATIONS

This application claims the benefit of priority to U.S. Provisional Patent Application No. 63/750,055, filed Jan. 27, 2025, the contents of which is hereby incorporated by reference herein.

### BACKGROUND OF THE INVENTION

Suicide is indeed a serious public health problem with far-reaching impacts. In 2021, suicide was responsible for 48,183 deaths in the United States, about one death every 11 minutes (Centers for Disease Control and Prevention, “Suicides increased in 2021, reaching highest level since 2018: CDC report,” ABC News, 2023). That same year, an estimated 12.3 million American adults seriously thought about suicide, 3.5 million planned a suicide attempt, and 1.7 million attempted suicide (Centers for Disease Control and Prevention, “Facts About Suicide,” 2023).

Suicide affects people of all ages but is particularly impactful among certain groups. In 2021, it was among the top 9 leading causes of death for people ages 10-64 and the second leading cause of death for those ages 10-14 and 20-34 (Centers for Disease Control and Prevention, “Suicide Mortality in the United States, 2001-2021,” 2023). Suicide rates vary by race/ethnicity, with the highest rates among non-Hispanic American Indian/Alaska Native people followed by non-Hispanic White people (American Foundation for Suicide Prevention, “Suicide statistics,” 2024). Other groups with higher-than-average suicide rates include veterans, people living in rural areas, and workers in certain industries like mining and construction (Centers for Disease Control and Prevention, “Suicide Rates by Industry and Occupation National Vital . . . ,” 2023). Young people who identify as lesbian, gay, or bisexual have a higher prevalence of suicidal thoughts and behavior compared to their heterosexual peers (The Trevor Project, “New Research on LGBTQ+ Teen Suicide Rates,” Newport Academy, 2024).

The impact of suicide extends far beyond the individual. Survivors of suicide attempts may experience serious injuries with long-term health effects, as well as depression and other mental health concerns. The effects on friends, loved ones, coworkers, and the community can be profound, including prolonged grief, shock, anger, guilt, symptoms of depression or anxiety, and even thoughts of suicide themselves. The financial toll is also significant, with suicide and nonfatal self-harm costing the nation over $510 billion in medical costs, work loss costs, value of statistical life, and quality of life costs in 2020 (Florence et al., “Economic Cost of U.S. Suicide and Nonfatal Self-harm,” PubMed Central, 2024).

Many factors can influence suicide risk. People who have experienced violence, including child abuse, bullying, or sexual violence have a higher suicide risk (Mondin et al., “Sexual violence, mood disorders and suicide risk,” SciELO, 2024). Conversely, being connected to family and community support and having easy access to healthcare can decrease suicidal thoughts and behaviors (Centers for Disease Control and Prevention, “Facts About Suicide,” 2023).

Current treatments for suicidal ideation and behavior have limitations. While antidepressants and antipsychotics can be effective for some patients, up to 75% experience side effects that may impair quality of life, contribute to poor medication adherence, and increase risk of relapse (Velligan et al., “Antipsychotic Treatment Failure: A Systematic Review on Risk Factors and Interventions,” Frontiers in Neuroscience, 2020). Additionally, about 9% of patients with depression and 73% of patients with first-episode psychosis switch or discontinue their initial medication, often due to intolerable side effects or inadequate response (Patteet et al., “Evidence-Based Applications of Combination Psychotherapy and Pharmacotherapy for Depression,” PubMed Central, 2016).

There is a need for improved treatment options that can effectively reduce suicidal ideation and behavior while minimizing side effects and the need for medication changes. Combination therapies using agents with complementary mechanisms of action may offer advantages over monotherapy in managing complex psychiatric conditions (Howes et al., “The Advantages of Combining Therapies in Treating Psychiatric Disorders,” PubMed Central, 2024). However, combination therapies can heighten the problems of the individual monotherapies (e.g., side effects that may impair quality of life, contribute to poor medication adherence, and increase risk of relapse) (Patteet et al., “Evidence-Based Applications of Combination Psychotherapy and Pharmacotherapy for Depression,” PubMed Central, 2016).

### SUMMARY OF THE INVENTION

The inventors have discovered a novel dosing regimen for the combination of nefazodone hydrochloride and risperidone in a single dosage form that provides significant advantages in treating serious psychiatric disorders, particularly those involving suicidal ideation. This combination therapy achieves the desired therapeutic effect while mitigating side effects and addressing individual variability, pharmacokinetics, and safety considerations.

The dosing regimen, which includes an initial dosing phase, titration phase, and maintenance phase, allows for a carefully controlled increase in medication levels. This approach surprisingly enables patients to achieve optimal therapeutic benefits while minimizing adverse effects, leading to improved quality of life and treatment adherence. The ability to fine-tune dosages of both medications independently during the titration phase is particularly advantageous, as it allows for personalized treatment tailored to each patient's unique response.

Perhaps most notably, this combination therapy in a single dosage form represents a significant advancement in treating complex psychiatric conditions involving suicidal ideation. The synergistic effects of nefazodone hydrochloride and risperidone, when administered according to this specific regimen, appear to provide more comprehensive symptom relief than either medication alone. This unexpected result offers new hope for patients who have not responded adequately to existing monotherapies or other combination treatments.

The inventors have discovered that the combination of nefazodone hydrochloride and risperidone, when administered according to the specific dosing regimen described herein, produces unexpected synergistic effects in treating psychiatric disorders involving suicidal ideation, while mitigating side effects. This synergistic effect is evidenced by a greater than additive reduction in suicidal thoughts and behaviors compared to what would be expected from the individual effects of nefazodone and risperidone alone. Specifically, patients treated with the combination therapy showed a reduction in suicidal ideation scores on standardized assessments, significantly higher than the reduction with each of nefazodone monotherapy and risperidone monotherapy. Furthermore, the combination therapy resulted in a significant decrease in suicide attempts, whereas monotherapy with either drug alone showed no significant reduction. This synergistic effect could not have been predicted based on the known properties of the individual drugs, and provides a significant and unexpected therapeutic benefit for patients with treatment-resistant depression and chronic suicidal ideation. Additionally, the carefully titrated dosing regimen described herein allows for optimal therapeutic effects while minimizing adverse reactions, resulting in improved treatment adherence and outcomes.

The present invention provides for a method of treating a person suffering from a psychiatric disorder. The method includes orally administering to the person a unit dose containing nefazodone hydrochloride, risperidone, and one or more pharmaceutical excipients. The administration includes a dosing regimen that includes an initial dosing, a titration, and maintenance dosing. Within each, a unit dose including nefazodone hydrochloride, risperidone, and one or more pharmaceutical excipients is orally administered to the subject, BID. The initial dosing typically includes a daily administration of 0.375±0.125 mg risperidone and 50±25 mg nefazodone hydrochloride. The initial dosing is typically carried out for at least 1 week. The titration (or dosing escalation) typically includes increasing the nefazodone hydrochloride in increments of 100±50 mg per day, until the maintenance dose is achieved. The titration also typically includes increasing the risperidone in increments of 0.375±0.125 mg per day, until the maintenance dose is achieved. The titration typically occurs at intervals of once per at least 1 week and over a period of time of, e.g., at least 2 weeks. The titration of risperidone is independent of the titration of nefazodone hydrochloride. Specifically, at any increment during the titration, (i) the dose of risperidone (but not nefazodone hydrochloride) can increase, (ii) the dose of nefazodone hydrochloride (but not risperidone) can increase, or (iii) the dose of both risperidone and nefazodone hydrochloride can increase. In any event, the titration is carried out until the maintenance dose is achieved for both risperidone and nefazodone hydrochloride. The maintenance dosing is achieved at a daily dose of 500±125 mg nefazodone hydrochloride and 1.75±0.5 mg risperidone. Each dosing is carried out twice daily (BID) with a solid unit dose. Within the course of the method of treatment, the person can be monitored, e.g., at least once per week by a medical professional for observation of emerging suicidal thoughts or behaviors.

The present invention provides for a method of treating a person suffering from a psychiatric disorder. The method includes orally administering to the person a unit dose containing nefazodone hydrochloride, risperidone, and one or more pharmaceutical excipients. The administration includes another dosing regimen that includes an initial dosing, a titration, and maintenance dosing. Within each, a unit dose including nefazodone hydrochloride, risperidone, and one or more pharmaceutical excipients is orally administered to the subject, BID. The initial dosing typically includes a daily administration of 0.375±0.125 mg risperidone and 50±25 mg nefazodone hydrochloride. The initial dosing is carried out for 2 week. The titration (or dosing escalation) includes increasing the nefazodone hydrochloride in increments of 100 mg per day, until the maintenance dose is achieved. The titration also includes increasing the risperidone in increments of 0.5 mg per day, until the maintenance dose is achieved. The titration occurs at intervals of once per week and over a period of time of, e.g., 3-5 weeks. The titration of risperidone is independent of the titration of nefazodone hydrochloride. Specifically, at any increment during the titration, (i) the dose of risperidone (but not nefazodone hydrochloride) can increase, (ii) the dose of nefazodone hydrochloride (but not risperidone) can increase, or (iii) the dose of both risperidone and nefazodone hydrochloride can increase. In any event, the titration is carried out until the maintenance dose is achieved for both risperidone and nefazodone hydrochloride. The maintenance dosing is achieved at a daily dose of 500±100 mg nefazodone hydrochloride and 1.75±0.5 mg risperidone. Each dosing is carried out twice daily (BID) with a solid unit dose. Within the course of the method of treatment, the person can be monitored, e.g., at least once per week by a medical professional for observation of emerging suicidal thoughts or behaviors.

The present invention provides for a method of treating a person suffering from a psychiatric disorder. The method includes orally administering to the person a unit dose containing nefazodone hydrochloride, risperidone, and one or more pharmaceutical excipients. The administration includes another dosing regimen that includes an initial dosing, a titration, and maintenance dosing. Within each, a unit dose including nefazodone hydrochloride, risperidone, and one or more pharmaceutical excipients is orally administered to the subject, BID. The initial daily dose includes (i) 0.25 mg risperidone and 50 mg nefazodone hydrochloride, or (ii) 0.50 mg risperidone and 50 mg nefazodone hydrochloride, administered in divided doses twice daily, in a solid oral dosage form, for a period of time of up to 2 weeks. The daily dose of 0.25 mg risperidone and 50 mg nefazodone hydrochloride is administered BID as a solid oral dosage form including 0.125 mg risperidone and 25 mg nefazodone hydrochloride. The daily dose of 0.25 mg risperidone and 50 mg nefazodone hydrochloride is administered BID as a solid oral dosage form including 0.125 mg risperidone and 25 mg nefazodone hydrochloride. The daily dose of 0.50 mg risperidone and 50 mg nefazodone hydrochloride is administered BID as a solid oral dosage form including 0.25 mg risperidone and 25 mg nefazodone hydrochloride. The titrated daily includes, sequentially, (i) 0.75 mg risperidone and 150 mg nefazodone hydrochloride, 1.25 mg risperidone and 250 mg nefazodone hydrochloride, 1.5 mg risperidone and 350 mg nefazodone hydrochloride, and 1.5 mg risperidone and 400 mg nefazodone hydrochloride, or (ii) 1.0 mg risperidone and 150 mg nefazodone hydrochloride, 1.5 mg risperidone and 250 mg nefazodone hydrochloride, 1.5 mg risperidone and 350 mg nefazodone hydrochloride, and 1.5 mg risperidone and 400 mg nefazodone hydrochloride, administered in divided doses twice daily, in a solid oral dosage form. The titrated dosing is carried out for a period of time of 2-5 weeks. The daily dose of 0.75 mg risperidone and 150 mg nefazodone hydrochloride is administered BID as a solid oral dosage form including 0.375 mg risperidone and 75 mg nefazodone hydrochloride. The daily dose of 1.25 mg risperidone and 250 mg nefazodone hydrochloride is administered BID as a solid oral dosage form including 0.625 mg risperidone and 125 mg nefazodone hydrochloride. The daily dose of 1.5 mg risperidone and 350 mg nefazodone hydrochloride is administered BID as a solid oral dosage form including 0.75 mg risperidone and 175 mg nefazodone hydrochloride. The daily dose of 1.5 mg risperidone and 400 mg nefazodone hydrochloride is administered BID as a solid oral dosage form including 0.75 mg risperidone and 200 mg nefazodone hydrochloride. The daily dose of 1.0 mg risperidone and 150 mg nefazodone hydrochloride is administered BID as a solid oral dosage form including 0.5 mg risperidone and 75 mg nefazodone hydrochloride. The daily dose of 1.5 mg risperidone and 250 mg nefazodone hydrochloride is administered BID as a solid oral dosage form including 0.75 mg risperidone and 125 mg nefazodone hydrochloride. The daily dose of 1.5 mg risperidone and 350 mg nefazodone hydrochloride is administered BID as a solid oral dosage form including 0.75 mg risperidone and 175 mg nefazodone hydrochloride. The daily dose of 1.5 mg risperidone and 400 mg nefazodone hydrochloride is administered BID as a solid oral dosage form including 0.75 mg risperidone and 200 mg nefazodone hydrochloride. The maintenance daily dose includes 1.5-2 mg risperidone and 400-600 mg nefazodone hydrochloride. Each dosing is carried out twice daily (BID) in a solid unit dose. Within the course of the method of treatment, the person can be monitored, e.g., at least once per week by a medical professional for observation of emerging suicidal thoughts or behaviors.

The present invention also provides for an oral unit dosage form that includes one or more pharmaceutically acceptable excipients and: (a) 0.1875±0.065 mg risperidone and 25±5 mg nefazodone hydrochloride; (b) 0.55±0.3 mg risperidone and 125±75 mg nefazodone hydrochloride; or (c) 0.875±0.125 mg risperidone and 250±mg nefazodone hydrochloride.

The present invention also provides for a therapeutic package that includes: (a) a finished pharmaceutical container with printed indicia located thereon; and (b) multiple oral unit dosages contained within the finished pharmaceutical container; wherein, the oral unit dosages include: (i) multiple oral unit dosages, each independently including 0.1875±0.065 mg risperidone and 25±5 mg nefazodone hydrochloride; (ii) multiple oral unit dosages, each independently including 0.55±0.3 mg risperidone and 125±75 mg nefazodone hydrochloride; and/or (iii) multiple oral unit dosages, each independently including 0.875±0.125 mg risperidone and 250±mg nefazodone hydrochloride.

### DETAILED DESCRIPTION OF THE INVENTION

The present invention relates to the psychotherapeutic combination of (i) nefazodone, or a pharmaceutically acceptable salt thereof, and (ii) risperidone. The psychotherapeutic combination is present within the same unit dose, along with (iii) one or more pharmaceutically acceptable excipients. The unit doses can be contained within a finished pharmaceutical container. Together with written matter or printed indicia, the finished pharmaceutical container can optionally be contained within an outside container. The unit dose can be administered to a person for the treatment of a psychiatric disorder. The psychiatric disorder can include, e.g., suicidal behavior, suicidal ideation, and/or major depressive disorder (MDD). This includes persons at risk of committing suicide, persons having recently attempted to commit suicide, and/or persons having a history of suicide attempts.

The use of the terms “a” and “an” and “the” and similar referents in the context of describing the invention are to be construed to cover both the singular and the plural, unless otherwise indicated herein or clearly contradicted by context.

The description herein of any aspect or aspect of the invention using terms such as “comprising,” “having,” “including” or “containing” with reference to an element or elements is intended to provide support for a similar aspect or aspect of the invention that “consists of,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations, and features of the invention mentioned herein may be claimed separately, or in any combination.

### Definitions

Throughout the description, the term “nefazodone” refers to refers to the compound 1-(3-[4-(3-chlorophenyl)piperazin-1-yl]propyl)-3-ethyl-4-(2-phenoxyethyl)-1H-1,2,4-triazol-5(4H)-one. The chemical structure is shown below.

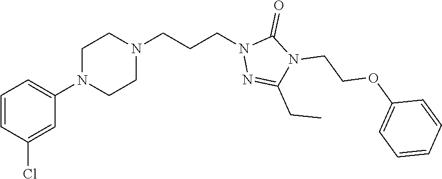 [SMILES: CCc1nn(CCCN2CCN(c3cccc(Cl)c3)CC2)c(=O)n1CCOc1ccccc1]

Nefazodone can also exist in the salt form (e.g., nefazodone hydrochloride). Nefazodone is an antidepressant and has the CAS number 83366-66-9 (82752-99-6 as the hydrochloride), unique ingredient identifier (UNII) 59H4FCV1TF, chemical formula C₂₅H₃₂ClN₅O₂, and molar mass 470.01 g·mol⁻¹. Nefazodone is a selective serotonin 5-HT₂ receptor antagonist. Preparation: D. L. Temple, Jr., W. G. Lobeck, Jr., U.S. Pat. No. 4,338,317 (1982 to Mead Johnson). Synthesis and x-ray crystal structure: G. D. Madding et al, J. Heterocycl. Chem. 22, 1121 (1985). Pharmacology: A. S. Eison et al., Psychopharmacol. Bull. 26, 311 (1990). HPLC determination in plasma: J. E. Franc et al., J. Chromatogr. 570, 129 (1991). Clinical trial in depression: M. F. D'Amico et al., Psychopharmacol. Bull. 26, 147 (1990); in combination with psychotherapy for chronic depression: M. B. Keller et al., N. Engl. J. Med. 342, 1462 (2000). Review: W. E. Heydorn, Expert Opin. Invest. Drugs 4, 131-137 (1995). The FDA (3Q 2022) provides for the following drug master file (DMF) holders: Omnichem SA (Nefazodone II), Esteve Quimica SA (Nefazodone Hydrchloride), TEVA Pharmaceutical Industries LTD (Nefazodone Hydrochloride USP), and Signa SA DE CV (Nefazodone Hydrochloride). Throughout the description, the term “nefazodone,” without specification of any particular salt form, is intended to include any form of the compound, such as the free base and pharmaceutically acceptable salts. The free base and pharmaceutically acceptable salts include anhydrous forms and solvated forms such as hydrates. The anhydrous forms and the solvates include amorphous and crystalline forms. In a particular embodiment, nefazodone is in the form of the hydrochloride salt and is denoted “nefazodone hydrochloride” or “nefazodone HCl”.

Throughout the description, the term “risperidone” refers to the compound 3-[2-[4-(6-fluoro-1,2-benzoxazol-3-yl)piperidin-1-yl]ethyl]-2-methyl-6,7,8,9-tetrahydropyrido[1,2-a]pyrimidin-4-one. The chemical structure is shown below.

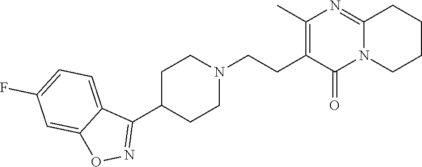 [SMILES: Cc1nc2n(c(=O)c1CCN1CCC(c3noc4cc(F)ccc34)CC1)CCCC2]

Risperidone is an antipsychotic and has the CAS number 106266-06-2, unique ingredient identifier (UNII) L6UH7ZF8HC, chemical formula C₂₃H₂₇FN₄O₂, and molar mass 410.493 g·mol⁻¹. Risperidone is a combined serotonin (5-HT₂) and dopamine (D₂) receptor antagonist. Preparation: L. E. J. Kennis, J. Vandenberk, EP 196132; eidem, U.S. Pat. No. 4,804,663 (1986, 1989 both to Janssen). Pharmacology: P. A. J. Janssen et al., T. Pharmacol. Exp. Ther. 244, 685 (1988). Receptor binding studies: J. E. Leysen et al., ibid. 247, 661 (1988). HPLC determination in plasma: A. Avenoso et al., J. Chromatogr. B 746, 173 (2000). Clinical study in psychoses: Y. G. Gelders et al., Pharmacopsychiatry 23, 206 (1990); in autism: L. Scahill et al., N. Engl. J. Med. 347, 314 (2002). Brief review: M. G. Livingston, Lancet 343, 457-460 (1994). Review of pharmacology and therapeutic potential: S. Grant, A. Fitton, Drugs 48, 253-273 (1994); B. Green, Curr. Med. Res. Opin. 16, 57-65 (2000); of clinical experience in schizophrenia: H.-J. Moller, Expert Opin. Pharmacother. 6, 803-818 (2005). The FDA (3Q 2022) provides for the following drug master file (DMF) holders: Janssen Pharmaceutica NV (Risperidone (R064766) Drug Substance), TEVA Pharmaceutical Industries LTD (Risperidone USP), INKE SA (Risperidone), Dr. Reddy's Laboratories LTD (Risperidone EP), and Medichem SA (Risperidone). Throughout the description, the term “risperidone,” without specification of any particular physical form, is intended to include any form of the compound, such as anhydrous forms and solvated forms such as hydrates. The anhydrous forms and the solvates include amorphous and crystalline forms.

In the present context, the terms “psychiatric disorder” and “mental disorder” encompass a wide range of conditions characterized by significant disturbances in cognition, emotion regulation, or behavior that reflect a dysfunction in psychological, biological, or developmental processes underlying mental functioning. These disorders are defined and classified in the DSM-5-TR (Diagnostic and Statistical Manual of Mental Disorders, 5th Edition, Text Revision; American Psychiatric Association) and include, but are not limited to:
1. Schizophrenia spectrum and other psychotic disorders
2. Depressive disorders (e.g., major depressive disorder)
3. Bipolar and related disorders
4. Anxiety disorders
5. Obsessive-compulsive and related disorders
6. Trauma- and stressor-related disorders
7. Neurodevelopmental disorders
8. Personality disorders

These disorders may be accompanied by suicidal behavior and/or suicidal ideation. The duration of a psychiatric disorder can vary:
1. Acute: lasting for a finite period of time
2. Chronic: persistent or long-lasting, potentially lifelong
3. Episodic: recurring episodes with periods of remission in between

The severity, course, and impact on functioning can differ significantly among individuals and may change over time.

In the present context, the term “suicidal behavior” refers to actions that intentionally put oneself at risk of death. Suicidal behavior encompasses a range of actions, from preparatory behaviors to fatal outcomes. The Columbia-Suicide Severity Rating Scale (C-SSRS), a widely used assessment tool, categorizes suicidal behavior into five subtypes:
1. Completed suicide: A self-injurious act that results in death, with evidence of intent to die.
2. Suicide attempt: A potentially self-injurious act committed with at least some intent to die, which may or may not result in injury.
3. Interrupted attempt: When a person is stopped by external circumstances from starting a potentially self-injurious act they intended to carry out.
4. Aborted attempt: When a person begins to take steps toward making a suicide attempt but stops themselves before engaging in any self-destructive behavior.
5. Preparatory actions toward imminent suicidal behaviors: Acts or preparation indicating intent for a suicide attempt (e.g., collecting pills, purchasing a weapon), without initiating a suicide attempt.

In the present context, the term “completed suicide” refers to a type of suicidal behavior in which an individual dies as a result of self-inflicted injury with evidence of intent to end one's life. This intent may be explicitly stated or inferred from the circumstances and nature of the self-injurious act. Completed suicide is distinguished from other forms of suicidal behavior by its fatal outcome and the presence of suicidal intent.

In the present context, the term “suicide attempt” refers to a type of suicidal behavior involving a potentially self-injurious act associated with at least some intent to die. The intent to end one's life, to any degree, may be explicitly stated or inferred from the individual's behavior or circumstances. A suicide attempt may or may not result in actual physical injury.

In the present context, the term “interrupted suicide attempt” refers to a type of suicidal behavior in which a person is stopped by external circumstances from initiating a potentially self-injurious act intended to result in death. The interruption occurs before the person can begin the self-destructive behavior, preventing what would have otherwise been an actual suicide attempt. This differs from an aborted attempt, where the person stops themselves, as the interruption comes from an outside source or event.

In the present context, the term “aborted suicide attempt” refers to a type of suicidal behavior in which a person begins to take steps toward making a suicide attempt but stops themselves before engaging in any self-destructive behavior. This differs from an interrupted attempt, where external circumstances prevent the act. In an aborted attempt, the individual voluntarily ceases their actions without intervention from others or external events. Examples may include:
Obtaining means for suicide but deciding not to use them
Going to a location to attempt suicide but leaving on one's own accord
Writing a suicide note but choosing not to take further action
Beginning to engage in self-harm behavior but stopping oneself before causing injury

The key distinguishing feature is that the person autonomously decides to stop the attempt, rather than being prevented by outside factors.

In the present context, the term “preparatory acts toward imminent suicidal behaviors” refers to actions taken to prepare for an attempt on one's life, but before starting the potentially self-injurious behavior. These acts may include, but are not limited to:
Collecting and assembling means for suicide (e.g., obtaining pills, purchasing a weapon)
Preparing for one's death (e.g., writing a suicide note, giving away possessions)
Researching suicide methods
Making arrangements for the act (e.g., choosing a time and place)
Rehearsing or practicing aspects of the attempt

These preparatory acts indicate a serious risk of imminent suicidal behavior and should be taken as warning signs requiring immediate intervention. It's important to note that these acts are distinct from non-suicidal self-injury or self-harm behaviors, which are addressed separately.

In the present context, the term “suicidal ideation” or “suicidal thoughts” refers to a person having thoughts, ideas, or ruminations about the possibility of ending one's own life. The DSM-5 defines it as “thoughts about self-harm, with deliberate consideration or planning of possible techniques of causing one's own death”. The Centers for Disease Control and Prevention defines suicidal ideation as “thinking about, considering, or planning suicide”. The ICD-11 describes suicidal ideation as “thoughts, ideas, or ruminations about the possibility of ending one's life, ranging from thinking that one would be better off dead to formulation of elaborate plans”.

Suicidal ideation is not a diagnosis but a symptom that can occur in various mental disorders or in response to adverse life events. The severity ranges from fleeting thoughts to detailed planning. Passive suicidal ideation involves thoughts about not wanting to live, while active suicidal ideation involves considering specific methods or forming plans to die.

While suicidal ideation is strongly associated with mood disorders, particularly depression, it can occur in numerous other psychiatric conditions. Mental health experts recommend treatment for individuals with suicidal ideation, regardless of diagnosis, due to the risk of suicidal acts and associated problems.

Disorders associated with increased risk of suicidal ideation include:
Mood disorders (major depressive disorder, dysthymia, bipolar disorder)
Anxiety disorders
Psychotic disorders (schizophrenia, schizoaffective disorder)
Personality disorders (especially borderline personality disorder)
Trauma-related disorders (PTSD, complex PTSD)
Substance use disorders
Neurodevelopmental disorders (autism spectrum disorder, ADHD)
Other conditions (e.g., body dysmorphic disorder, gender dysphoria, eating disorders)

The Columbia-Suicide Severity Rating Scale (C-SSRS) defines five levels of suicidal ideation:
1. Passive
2. Active: nonspecific (no method, intent, or plan)
3. Active: method, but no intent or plan
4. Active: method and intent, but no plan
5. Active: method, intent, and plan

In the present context, the term “passive suicidal ideation: wish to be dead” refers to a type of suicidal ideation characterized by thoughts of death or a desire not to be alive, without active intentions or plans to end one's life. This may include:
Wishing to be dead or not alive anymore
Hoping to fall asleep and not wake up
Feeling that others would be better off if the person were dead
Thoughts that life is not worth living

These thoughts are considered passive because they do not involve specific plans or intent to act on suicidal urges. However, passive suicidal ideation is still a serious clinical concern that warrants professional evaluation and intervention, as it may progress to more active forms of suicidal ideation or behavior.

In the present context, the term “active suicidal ideation: nonspecific (no method, intent, or plan)” refers to a type of suicidal ideation characterized by general thoughts of wanting to end one's life or commit suicide, without specific methods, intent, or plans. Examples include:
Thoughts like “I've thought about killing myself” or “I wish I were dead”
Fleeting or recurring ideas about suicide without concrete details
Desire to die or stop living, but without considering how to act on it
Vague notions of suicide that lack any particular course of action

This form of suicidal ideation is considered active because the person is explicitly thinking about suicide, but nonspecific as it lacks the detailed planning or intent found in more severe forms. It is important to note that even nonspecific suicidal thoughts can indicate significant distress and should be taken seriously in clinical assessment.

In the present context, the term “active suicidal ideation: method, but no intent or plan” refers to a type of suicidal ideation in which the person has thoughts of suicide and has considered at least one method during the assessment period. This differs from having a specific plan with detailed time, place, or method considerations. It includes situations where a person might say, “I've thought about taking an overdose, but I haven't made any specific plans about when, where, or how I would do it, and I don't intend to go through with it.”

In the present context, the term “active suicidal ideation: method and intent, but no plan” refers to a type of suicidal ideation characterized by:
1. Active thoughts of killing oneself
2. Consideration of at least one method for suicide
3. Some degree of intent to act on these thoughts
4. Absence of a specific plan for suicide

This differs from passive suicidal ideation or thoughts without intent, as the person expresses some willingness to act on their suicidal thoughts. However, it is distinguished from more severe forms of suicidal ideation by the lack of a detailed plan.

In the present context, the term “active suicidal ideation: method, intent, and plan” refers to a type of suicidal ideation characterized by:
1. Thoughts of killing oneself
2. A specific method for suicide that has been identified
3. Intent to act on these thoughts
4. A plan with details that are fully or partially worked out

This is considered the most severe form of suicidal ideation, as it combines specific thoughts about suicide methods with both intent and planning. The presence of a plan implies some degree of intent, but the level of intent may vary. This type of ideation represents an acute risk that requires immediate clinical intervention.

In the present context, the term “major depressive disorder” or “MDD” (also known as clinical depression) refers to a psychiatric disorder characterized by at least two weeks of persistent symptoms that significantly impact daily functioning. The core symptoms are:
1. Depressed mood
2. Loss of interest or pleasure in activities (anhedonia)

Additional symptoms may include:
Changes in appetite or weight
Sleep disturbances
Psychomotor agitation or retardation
Fatigue or loss of energy
Feelings of worthlessness or excessive guilt
Difficulty concentrating or indecisiveness
Recurrent thoughts of death or suicide

Diagnosis is based on:
Patient's reported experiences
Observations by relatives or friends
Mental status examination

The course can vary from a single episode to recurrent episodes throughout life. Diagnostic criteria are outlined in the DSM-5 and ICD-11, which provide specifiers for:
Severity (mild, moderate, severe)
Presence of psychotic features
Remission status
Pattern (single episode or recurrent)

MDD is classified under “Depressive Disorders” in the broader category of “Mood Disorders.” For a diagnosis, symptoms must cause significant distress or impairment in social, occupational, or other important areas of functioning.

In the present context, the term “major depressive episode” refers to a period characterized by symptoms of major depressive disorder, as defined in psychiatric diagnostic criteria such as the DSM-5 and ICD-11. Key features include:
1. Depressed mood for at least two weeks
2. Loss of interest or pleasure in most activities

Additional symptoms may include:
Feelings of emptiness, hopelessness, or worthlessness
Excessive guilt or anxiety
Irritability
Significant changes in appetite or weight
Sleep disturbances (insomnia or hypersomnia)
Fatigue or loss of energy
Difficulty concentrating or making decisions
Psychomotor agitation or retardation
Recurrent thoughts of death or suicide

Physical symptoms may also be present, such as unexplained aches, pains, or digestive problems that are resistant to treatment.

To meet diagnostic criteria, symptoms must cause significant distress or impairment in social, occupational, or other important areas of functioning, and not be attributable to the effects of a substance or another medical condition.

In the present context, the term “treatment-resistant depression” refers to a form of major depressive disorder that does not respond adequately to at least two different antidepressant medications given at adequate doses for an adequate duration. This condition affects up to 30% of patients with major depressive disorder and may have different underlying pathophysiological mechanisms compared to treatment-responsive depression. While some studies have found combination antidepressant therapy to be more effective than monotherapy for treatment-resistant depression, particularly combinations including a reuptake inhibitor with an α2-adrenergic antagonist, other research has shown mixed results. The optimal treatment approach remains unclear and may need to be tailored to the individual patient, weighing potential benefits against the risk of increased side effects with combination therapy.

In the present context, the term “effective amount” or “therapeutically effective amount” of therapeutic agent(s) refers to:
1. A nontoxic but sufficient quantity to produce the desired therapeutic effect
2. An amount that alleviates, arrests (partially or fully), removes, or delays the clinical manifestations of a given psychiatric disorder and its complications
3. For combination therapy, the amount of each component that provides the desired effect when used together

The effective amount depends on factors such as:
Severity of the disorder
Patient's weight and general health condition
Individual patient response

Determining the appropriate dosage involves:
Experimentation;
Constructing a matrix of values
Testing different points in the matrix

This process falls within skills of a sufficiently trained and experienced physician.

In the present context, the term “pharmaceutical product” or “combination product” refers to any product containing:
1. Nefazodone, or a pharmaceutically acceptable salt thereof
2. Risperidone
3. One or more pharmaceutically acceptable excipients

The pharmaceutical product is typically in the form of a unit dose, such as:
Tablets
Capsules
Oral soluble films
Other solid oral dosage forms

The pharmaceutical product is preferably in a unitary dosage form suitable for oral administration. While other routes of administration are possible, oral administration is the primary focus for this combination product.

In the present context, the term “unit dose” or “unit dosage form” refers to physically discrete units of a pharmaceutical product described herein, each containing a predetermined quantity of:
1. Nefazodone, or a pharmaceutically acceptable salt thereof
2. Risperidone
3. One or more pharmaceutical excipients

These units are designed to deliver a specific dose calculated to produce the desired therapeutic effect. The unit dose format is advantageous for ease of administration and uniformity of dosage.

Unit doses may include, but are not limited to:
Solid oral dosage forms:
   Tablets (including scored tablets)
   Capsules (including gel capsules)
   Oral soluble films
   Lozenges
   Pills
   Granulates
   Powders
Liquid dosage forms:
   Syrups
   Aqueous or non-aqueous solutions
   Suspensions

While the primary focus is on oral administration, other forms such as sterile injectable solutions may also be considered unit doses in certain contexts.

In the present context, the term “pharmaceutically acceptable salts” refers to salts of the active compounds that are safe and effective for use in humans. These include:
1. Acid addition salts formed with organic or inorganic acids.
2. Addition salts of free bases.

Examples of acids that may form pharmaceutically acceptable acid addition salts include:
Organic acids:
Carboxylic acids: maleic, fumaric, benzoic, ascorbic, succinic, acetic, propionic, tartaric, salicylic, citric, lactic, malic, mandelic, cinnamic, aspartic, stearic, palmitic, glycolic, glutamic
Sulfonic acids: methanesulfonic, ethanedisulfonic, benzenesulfonic
Other organic acids: oxalic, embonic, p-aminobenzoic, itaconic
Inorganic acids:
Hydrochloric, hydrobromic, sulfuric, sulfamic, phosphoric, nitric

The term “pharmaceutically acceptable salts” also includes salts formed with modified nucleic acids, such as theophylline acetic acid and 8-halotheophyllines (e.g., 8-bromotheophylline).

In the present context, the term “antipsychotic drug” (also known as “neuroleptic”) refers to a class of psychotropic medications primarily used to manage psychosis and related disorders. These drugs are characterized by their ability to:
1. Manage positive symptoms of psychosis, including:
   Delusions
   Hallucinations
   Paranoia
   Disordered thought
2. Treat a range of conditions, such as:
   Schizophrenia
   Schizoaffective disorder
   Bipolar disorder (acute mania, mixed, and depressive episodes)
   Psychotic depression
   Treatment-resistant depression

Antipsychotics are classified into two main categories:
1. Typical (first-generation) antipsychotics
2. Atypical (second-generation) antipsychotics

These medications are often used in combination with mood stabilizers, particularly in the treatment of bipolar disorder. Their mechanism of action primarily involves modulating neurotransmitter systems, especially dopamine and serotonin pathways.

Antipsychotic drugs

| | |
|---|---|
| Generic Name | |
| (Active Ingredient) | Brand Name |

Typical antipsychotics

| | |
|---|---|
| Acepromazine | Atravet, Acezine |
| Acetophenazine | Tindal |
| Benperidol | Frenactyl |
| Bromperidol | Bromidol, Bromodol |
| Butaperazine | Repoise, Tyrylen |
| Carfenazine | |
| Chlorproethazine | |
| Chlorpromazine | Largactil, Thorazine |
| Chlorprothixene | Cloxan, Taractan, Truxal |
| Clopenthixol | Sordinol |
| Cyamemazine | Tercian |
| Dixyrazine | Esucos |
| Droperidol | Droleptan, Dridol, Inapsine, Xomolix, Innovar (+Fentanyl) |
| Fluanisone | |
| Flupentixol | Depixol, Fluanxol |
| Fluphenazine | Prolixin, Modecate |
| Fluspirilene | Redeptin, Imap |
| Haloperidol | Haldol |
| Levomepromazine | Nosinan, Nozinan, Levoprome |
| Lenperone | Elanone-V |
| Loxapine | Loxapac, Loxitane |
| Mesoridazine | Serentil |
| Metitepine | |
| Molindone | Moban |
| Moperone | Luvatren |
| Oxypertine | Equipertine, Forit, Integrin, Lanturil, Lotawin, Opertil |
| Oxyprothepine | Meclopin |
| Penfluridol | Semap, Micefal, Longoperidol |
| Perazine | Taxilan |
| Periciazine | Neuleptil, Neulactil |
| Perphenazine | Trilafon |
| Pimozide | Orap |
| Pipamperone | Dipiperon, Dipiperal, Piperonil, Piperonyl, Propitan |
| Piperacetazine | Quide |
| Pipotiazine | Piportil |
| Prochlorperazine | Compazine, Stemzine, Buccastem, Stemetil, Phenotil |
| Promazine | Sparine |
| Prothipendyl | |
| Spiperone | Spiroperidol, Spiropitan |
| Sulforidazine | Imagotan, Psychoson, Inofal |
| Thiopropazate | Artalan, Dartal, Dartalan, Dartan |
| Thioproperazine | Majeptil |
| Thioridazine | Mellaril, Melleril |
| Thiothixene | Navane |
| Timiperone | |
| Trifluoperazine | Stelazine |
| Trifluperidol | |
| Triflupromazine | Vesprin |
| Zuclopenthixol | Clopixol |

Atypical antipsychotics

| | |
|---|---|
| Amoxapine | Asendin, Asendis, Defanyl, Demolox |
| Amisulpride | Amazeo, Amipride, Amival, Solian, Soltus, Sulpitac, Sulprix |
| Aripiprazole | Abilify |
| Asenapine | Saphris |
| Blonanserin | Lonasen |
| Brexpiprazole | Rexulti |
| Cariprazine | Vraylar |
| Carpipramine | Prazinil, Defekton |
| Clocapramine | Clofekton, Padrasen |
| Clorotepine | Clotepin, Clopiben |
| Clotiapine | Entumine |
| Clozapine | Clozaril |
| Iloperidone | Fanapt |
| Levosulpiride | |
| Lumateperone | Caplyta |
| Lurasidone | Latuda |
| Melperone | Bunil, Buronil, Eunerpan |
| Mosapramine | Cremin |
| Nemonapride | Emilace |
| Olanzapine | Zyprexa, Ozace, Lanzek, Zypadhera |
| Paliperidone | Invega |
| Perospirone | Lullan |
| Quetiapine | Seroquel |
| Remoxipride | Roxiam |
| Reserpine | Raudixin, Serpalan, Serpasil |
| Risperidone | Risperdal, Zepidone |
| Sertindole | Serdolect |
| Sulpiride | Sulpirid, Eglonyl |
| Sultopride | Barnetil, Barnotil, Topral |
| Tiapride | Equilium, Tiapridal |
| Veralipride | Agreal, Agradil |
| Ziprasidone | Geodon, Zeldox |

In the present context, the term “antidepressant drug” refers to a medication primarily used to treat depression and related mood disorders. These drugs may also be prescribed for:
1. Major depressive disorder (MDD)
2. Anxiety disorders
3. Chronic pain conditions
4. Management of certain addictions

Antidepressants encompass several classes of medications, including:
1. Selective serotonin reuptake inhibitors (SSRIs)
2. Serotonin-norepinephrine reuptake inhibitors (SNRIs)
3. Serotonin modulators and stimulators (SMSs)
4. Serotonin antagonist and reuptake inhibitors (SARIs)
5. Norepinephrine reuptake inhibitors (NRIs)
6. Norepinephrine-dopamine reuptake inhibitors (NDRIs)
7. Tricyclic antidepressants (TCAs)
8. Tetracyclic antidepressants (TeCAs)
9. Monoamine oxidase inhibitors (MAOIs)

Each class of antidepressants works by modulating different neurotransmitter systems in the brain, primarily affecting serotonin, norepinephrine, and/or dopamine levels.

Antidepressant drugs

| | |
|---|---|
| Generic Name | |
| (Active ingredient) | Brand Name |

Selective serotonin reuptake inhibitors (SSRIs)

| | |
|---|---|
| Citalopram | (Celexa, Cipramil) |
| Escitalopram | (Lexapro, Cipralex) |
| Fluoxetine | (Prozac, Sarafem) |
| Fluvoxamine | (Luvox, Faverin) |
| Paroxetine | (Paxil, Seroxat) |
| Sertraline | (Zoloft, Lustral) |

Serotonin-norepinephrine reuptake inhibitors (SNRIs)

| | |
|---|---|
| Desvenlafaxine | (Pristiq) |
| Duloxetine | (Cymbalta) |
| Levomilnacipran | (Fetzima) |
| Milnacipran | (Ixel, Savella, Milnaneurax) |
| Venlafaxine | (Effexor, Trevilor) |

Serotonin modulators and stimulators (SMSs)

| | |
|---|---|
| Vilazodone | (Viibryd) |
| Vortioxetine | (Trintellix, Brintellix) |

Serotonin antagonist and reuptake inhibitors (SARIs)

| | |
|---|---|
| Trazodone | (Desyrel) |
| Nefazodone | (Dutonin, Nefadar, Serzone) - withdrawn/discontinued |
| Etoperidone | (Axiomin, Etonin) - withdrawn/discontinued |

Norepinephrine reuptake inhibitors (NRIs)

| | |
|---|---|
| Reboxetine | (Edronax) |
| Teniloxazine | (Lucelan, Metatone) - also a 5-HT_{2A} receptor antagonist |
| Viloxazine | (Vivalan) - also a 5-HT_{2B} receptor antagonist and 5-HT_{2C} |
| | receptor agonist |
| Atomoxetine | (Strattera) - Off-label only |

Norepinephrine-dopamine reuptake inhibitors (NDRIs)

| | |
|---|---|
| Bupropion | (Wellbutrin, Elontril) - also a non-competitive antagonist of |
| | nicotinic acetylcholine receptors |
| Amphetamines | (e.g., Adderall, Dexedrine, Vyvanse) - actually |
| | norepinephrine-dopamine releasing agents (NDRAs) - Off- |
| | label only |
| Methylphenidate | (Ritalin) - Off-label only |
| Modafinil | (Provigil) - actually a selective dopamine reuptake inhibitor |
| | plus other actions - Off-label only |
| Amineptine | (Survector, Maneon) |
| Nomifensine | (Merital, Alival) |

Tricyclic antidepressants (TCAs)

| | |
|---|---|
| Amitriptyline | (Elavil, Endep) |
| Amitriptylinexide | (Amioxid, Ambivalon, Equilibrin) |
| Clomipramine | (Anafranil) |
| Desipramine | (Norpramin, Pertofrane) |
| Dibenzepin | (Noveril, Victoril) |
| Dimetacrine | (Istonil) |
| Dosulepin | (Prothiaden) |
| Doxepin | (Adapin, Sinequan) |
| Imipramine | (Tofranil) |
| Lofepramine | (Lomont, Gamanil) |
| Melitracen | (Dixeran, Melixeran, Trausabun) |
| Nitroxazepine | (Sintamil) |
| Nortriptyline | (Pamelor, Aventyl) |
| Noxiptiline | (Agedal, Elronon, Nogedal) |
| Opipramol | (Insidon) |
| Pipofezine | (Azafen/Azaphen) |
| Protriptyline | (Vivactil) |
| Trimipramine | (Surmontil) |
| Amineptine | (Survector, Maneon) and tianeptine (Stablon, Coaxil) are |
| | technically TCAs but are atypical, and are grouped elsewhere. |
| Butriptyline | (Evadyne) - withdrawn/discontinued |
| Demexiptiline | (Deparon, Tinoran) - withdrawn/discontinued |
| Fluacizine | (Phtorazisin) - withdrawn/discontinued |
| Imipraminoxide | (Imiprex, Elepsin) - withdrawn/discontinued |
| Iprindole | (Prondol, Galatur, Tetran) - withdrawn/discontinued |
| Metapramine | (Timaxel) - withdrawn/discontinued |
| Propizepine | (Depressin, Vagran) - withdrawn/discontinued |
| Quinupramine | (Kinupril, Kevopril) - withdrawn/discontinued |
| Tiazesim | (Altinil) - actually not a TCA but a tricyclic-like antidepressant - |
| | withdrawn/discontinued |
| Tofenacin | (Elamol, Tofacine) - actually not a TCA but a tricyclic-like |
| | antidepressant - withdrawn/discontinued |

Tetracyclic antidepressants (TeCAs)

| | |
|---|---|
| Amoxapine | (Asendin) |
| Maprotiline | (Ludiomil) |
| Mianserin | (Tolvon) |
| Mirtazapine | (Remeron) |
| Setiptiline | (Tecipul) |

Monoamine oxidase inhibitors (MAOIs)
Irreversible
Non-selective

| | |
|---|---|
| Isocarboxazid | (Marplan) |
| Phenelzine | (Nardil) |
| Tranylcypromine | (Parnate) |
| Benmoxin | (Neuralex) - withdrawn/discontinued |
| Iproclozide | (Sursum) - withdrawn/discontinued |
| Iproniazid | (Marsilid) - withdrawn/discontinued |
| Mebanazine | (Actomol) - withdrawn/discontinued |
| Nialamide | (Niamid) - withdrawn/discontinued |
| Octamoxin | (Ximaol) - withdrawn/discontinued |
| Pheniprazine | (Catron) - withdrawn/discontinued |
| Phenoxypropazine | (Drazine) - withdrawn/discontinued |
| Pivhydrazine | (Tersavid) - withdrawn/discontinued |
| Safrazine | (Safra) - withdrawn/discontinued |

Selective for MAO-B

| | |
|---|---|
| Selegiline | (Eldepryl, Zelapar, Emsam) |

Reversible
Non-selective

| | |
|---|---|
| Caroxazone | (Surodil, Timostenil) - withdrawn/discontinued |

Selective for MAO-A

| | |
|---|---|
| Metralindole | (Inkazan) - sometimes described as reversible inhibitors of |
| | MAO-A (RIMAs) |
| Moclobemide | (Aurorix, Manerix) - sometimes described as reversible |
| | inhibitors of MAO-A (RIMAs) |
| Pirlindole | (Pirazidol) - sometimes described as reversible inhibitors of |
| | MAO-A (RIMAs) |
| Eprobemide | (Befol) - withdrawn/discontinued |
| Minaprine | (Brantur, Cantor) - withdrawn/discontinued |
| Toloxatone | (Humoryl) - withdrawn/discontinued |

Mixed
Non-selective

| | |
|---|---|
| Bifemelane | (Alnert, Celeport) - RIMA, irreversible inhibitor of MAO-B, |
| | and weak NRI |

In the present context, the terms “treating” and “treatment” refer to:
1. Reducing the severity and/or frequency of symptoms
2. Eliminating symptoms and/or their underlying cause
3. Preventing the occurrence of symptoms and/or their underlying cause
4. Improving or remediating damage caused by the condition

Treatment includes managing and caring for a patient to:
Alleviate
Arrest (partially or fully)
Remove
Delay the progress of the clinical manifestations of the psychiatric disorder

This encompasses:
1. Preventing a particular disorder or adverse physiological event in susceptible individuals
2. Treating clinically symptomatic individuals

The patient is typically a human, though treatment may also apply to other mammals.

In the present context, the terms “treatment of a psychiatric disorder” and “treating a psychiatric disorder” (and terms “treatment of a psychiatric disease” and “treating a psychiatric disease”) mean administering a pharmaceutical product described herein to a person who has a psychiatric disorder with the result that:
1. The symptoms of the psychiatric disorder are reduced in severity or frequency
2. The overall impact of the disorder on the person's functioning is mitigated
3. The progression of the disorder is slowed or halted
4. The risk of complications or associated conditions is decreased

This may lead to:
Improved quality of life for the person
Enhanced ability to engage in daily activities and social interactions
Reduced frequency or severity of acute episodes, if applicable
Potentially complete remission of symptoms in some cases

It's important to note that treatment outcomes can vary significantly between individuals, and complete prevention of all symptoms may not always be achievable for all psychiatric disorders.

In the present context, the terms “treatment of suicidal behavior” and “treating suicidal behavior” refer to administering a pharmaceutical product described herein to a person who exhibits suicidal behavior, with the intended result of:
1. Reducing the frequency or intensity of suicidal behaviors
2. Mitigating the risk factors associated with suicidal behaviors
3. Potentially preventing future suicide attempts or completed suicides

This treatment aims to:
Decrease the occurrence of specific suicidal actions
Improve the person's ability to cope with suicidal urges
Enhance protective factors against suicidal behavior
Address underlying mental health conditions contributing to suicidal behavior

The effectiveness of treatment may be measured by:
Reduced frequency or severity of suicidal thoughts or actions
Improved scores on standardized suicide risk assessment tools
Increased engagement with mental health support systems
Enhanced overall functioning and quality of life

It's important to note that while the goal is to prevent all suicidal behaviors, complete prevention may not always be achievable, and ongoing monitoring and support are typically carried out.

In the present context, the terms “treatment of suicidal ideation” and “treating suicidal ideation” refer to administering a pharmaceutical product described herein to a person experiencing suicidal thoughts, with the intended result of:
1. Reducing the frequency or intensity of suicidal thoughts
2. Mitigating the impact of suicidal thoughts on daily functioning
3. Potentially eliminating suicidal thoughts entirely

The effectiveness of treatment may be measured by:
Improved scores on standardized assessment tools, such as the MADRS item 10 or HAM-D items related to suicidal thoughts
Decreased frequency or severity of reported suicidal thoughts
Reduced risk of suicide attempts

It's important to note that while the goal is to prevent all suicidal thoughts and behaviors, complete elimination may not always be achievable for all patients. Ongoing monitoring and support are typically carried out as part of a comprehensive treatment plan.

In the present context, the terms “treatment of major depressive disorder” and “treating major depressive disorder” refer to administering a pharmaceutical product described herein to a person diagnosed with major depressive disorder, with the intended result of:
1. Reducing the severity of depressive symptoms
2. Decreasing the frequency of depressive episodes
3. Improving overall mood and functioning
4. Potentially achieving remission of depressive symptoms

The treatment aims to:
Alleviate core symptoms of depression (e.g., depressed mood, anhedonia, changes in sleep and appetite)
Enhance quality of life and daily functioning
Reduce the risk of relapse or recurrence
In some cases, prevent the onset of future depressive episodes

Effectiveness may be measured by:
Improved scores on standardized depression rating scales (e.g., MADRS, HAM-D)
Patient-reported improvements in mood and functioning
Clinician assessment of symptom reduction and overall improvement

It's important to note that while complete prevention of all depressive symptoms may be an ideal goal, it may not be achievable for all patients. Ongoing monitoring and support are typically part of a comprehensive treatment plan.

In the present context, the terms “subject,” “participant,” “research participant,” “patient,” and “individual” refer to a person (e.g., child, adolescent, or adult) who is diagnosed with or at risk for a psychiatric disorder. These terms may apply to a single person or a group of people involved in the research.

In the present context, the term “prescriber” refers to a licensed healthcare professional who has the legal authority to prescribe medications for the treatment of psychiatric disorders. This may include:
1. Physicians (MDs and DOs)
2. Psychiatrists
3. Nurse practitioners (NPs)
4. Physician assistants (PAs)
5. In some jurisdictions, clinical psychologists with additional training and certification

The prescriber is responsible for:
1. Evaluating the patient's condition
2. Determining the appropriate medication and dosage
3. Issuing a valid prescription
4. Monitoring the patient's response to treatment
5. Adjusting the treatment plan as needed

It's important to note that the specific professionals authorized to prescribe psychiatric medications may vary by jurisdiction and that prescribing authority is subject to applicable laws and regulations.

In the present context, the term “advertising” refers to communicating information about a pharmaceutical product to potential consumers or healthcare providers. This may include:
1. Notifying, informing, or apprising individuals about the product's efficacy, indications, or other attributes
2. Using various media channels, such as:
   Print media (newspapers, magazines)
   Digital media (internet advertisements, social media)
   Broadcast media (television or radio commercials)
   Outdoor media (billboard signs)
3. Providing information in product labeling or packaging
4. Distributing promotional materials to healthcare professionals

In the present context, the term “marketing” refers to the strategic process of promoting, selling, and distributing a product or service. This includes:
1. Market research and analysis
2. Product development and positioning
3. Pricing strategies
4. Promotional activities (advertising, public relations, social media campaigns)
5. Distribution channel management
6. Customer relationship management
7. Sales activities (including offers for sale and actual sales)
8. Post-sale customer support

Marketing aims to create value for customers and build strong customer relationships to capture value from customers in return. It encompasses all activities that connect a product or service with its target audience.

In the present context, the term “maintenance drugs” refers to medications prescribed to treat chronic, long-term conditions that are taken on a regular, recurring basis, often daily. These medications are typically used for ongoing management of persistent health issues and may be needed for months, years, or even a lifetime. Examples of conditions treated with maintenance drugs include depression, hypertension, diabetes, high cholesterol, asthma, and arthritis. Maintenance drugs are distinguished from acute medications, which are used for short-term conditions or symptoms. The goal of maintenance drug therapy is to control symptoms, prevent disease progression, and improve quality of life for individuals with chronic health conditions.
1. In the present context, the term “chronic” refers to a condition or state that:
2. Persists for an extended period, typically three months or longer
3. Develops slowly and/or is long-lasting
4. May have a pattern of recurrence or relapse
5. Often requires ongoing management or treatment

This term is often used in contrast to “acute” conditions, which are typically sudden in onset and short in duration. In the context of psychiatric disorders, chronic conditions may require long-term or indefinite treatment strategies.

In the present context, the term “long-term” refers to:
1. Extending over a significant duration, typically months or years
2. Occurring over an extended period, rather than a brief or acute timeframe
3. Involving ongoing or continuous treatment or management
4. Relating to effects, outcomes, or processes that persist or develop gradually over time

In the present context, the term “regular” refers to:
1. Occurring at fixed or predictable intervals
2. Following a consistent pattern or schedule
3. Happening with normal or expected frequency
4. Maintaining a relatively constant or uniform nature
5. Conforming to an established rule, principle, or pattern

In the present context, the term “continuous” refers to:
1. Occurring or existing without interruption
2. Unbroken in time or sequence
3. Forming an unbroken whole; without gaps or intervals
4. Unceasing in duration or extent

In the present context, the term “recurring” refers to:
1. Events or phenomena that happen repeatedly at regular or irregular intervals
2. Conditions or symptoms that return after a period of remission or absence
3. Actions or processes that are performed multiple times over a given period

In the present context, the term “initial dose” or “initial dosing” refers to the starting therapy in which the patient first receives the active ingredients (nefazodone hydrochloride and risperidone). This initial dosing:
1. Is administered in a specific amount over a defined period of time
2. For nefazodone hydrochloride:
   Typically starts at about 50 mg per day
   Is divided into two doses (25 mg twice daily)
   Is typically maintained for 1-2 weeks
3. For risperidone:
   Typically starts at 0.25-0.5 mg per day
   Is divided into two doses (0.125 mg or 0.25 mg, twice daily)
   Is typically maintained for 1-2 weeks

The initial dosing period allows for assessment of the patient's response and tolerability before any dose adjustments are made.

In the present context, “titration” or “dosing escalation” refers to the process of gradually increasing the dose of nefazodone hydrochloride and risperidone over time to achieve optimal therapeutic effect while minimizing side effects. The titration process is as follows:

For nefazodone hydrochloride:
Initial dose: Typically 50 mg per day, divided into two doses
Incremental increase: Usually about 50 mg per day
Interval between increases: No less than 1 week, as tolerated
Target therapeutic dose range: 400-600 mg per day

For risperidone:
Initial dose: Usually 0.25-0.5 mg per day
Incremental increase: Typically 0.25-0.75 mg per day
Interval between increases: No less than 1 week, as tolerated
Recommended maintenance dose range: 1.5-2 mg per day for most patients

The goal of titration is to identify the lowest effective dose that provides symptom relief while minimizing adverse effects. This individualized approach allows healthcare providers to adjust the medication regimen based on each patient's unique response and tolerability.

The initial dose, titration, and maintenance phases can be carried out under the supervision of qualified healthcare providers, which may include:
1. Psychiatrists
2. Primary care physicians
3. Psychiatric nurse practitioners
4. Physician assistants with mental health training

While some patients may require inpatient treatment in settings such as:
Psychiatric hospitals
Hospital psychiatric units
Residential treatment facilities

The majority of patients will typically receive initial dosing, titration, and maintenance treatment as outpatients. This may involve:
Regular office visits
Telemedicine appointments
Home health services in some cases

Ongoing monitoring and follow-up care in the community are significant components of the treatment plan, regardless of the initial setting.

In the present context, the terms “ongoing maintenance,” “maintenance therapy,” or “chronic therapy” refer to a treatment approach in which the patient receives long-term, continuous, and regular administration of a pharmaceutical product for managing a chronic disorder. This therapy involves maintaining a relatively consistent dose, referred to as a maintenance dose, to control symptoms, prevent disease progression, and improve overall quality of life.

In the present context, the term “finished pharmaceutical container” refers to devices designed for the storage and dispensing of pharmaceutical dosage forms. These include:
1. Containers for solid dosage forms:
   Bottles
   Blister packs
   Sachets
   Pill boxes
   Tablet dispensers
2. Containers for liquid dosage forms:
   Bottles
   Vials
   Ampoules
   Syringes
3. Specialized packaging:
   Safety packings
   Foil wrappings
   Medicine organizers
   Pill fobs and totes

These containers are designed to protect the integrity of the pharmaceutical product, ensure proper dosing, and in some cases, improve patient compliance. The specific type of container used depends on the nature of the dosage form, its stability requirements, and the intended use.

In the present context, the term “written matter” encompasses, but is not limited to:
1. Package inserts
2. Labels
3. Patient information leaflets
4. Instructions for use
5. Medication guides
6. Patient Package Inserts (PPIs)
7. Consumer medical information (CMI)
8. User manuals
9. Quick reference guides
10. Patient education materials
11. Digital content (e.g., websites, mobile apps)

This includes both physical and digital formats of information provided to patients, healthcare professionals, and other users of the pharmaceutical product. The content should be clear, accessible, and compliant with relevant regulatory requirements.

In the present context, the term “printed indicia” refers to markings on a pharmaceutical product or its packaging that may include:
1. Marketing company name
2. Manufacturing company name
3. Active ingredient name(s)
4. Brand name or trade name
5. Strength or concentration of active ingredient(s)
6. Dosage form (e.g., tablet, capsule, solution)
7. Route of administration (e.g., oral, topical, injectable)
8. Product serialization or lot number
9. Expiration date
10. Storage instructions
11. Warnings or precautions
12. Regulatory compliance symbols or codes

These markings are intended to provide significant information for identification, proper use, and traceability of the pharmaceutical product.

In the present context, the term “tablet” refers to a solid oral dosage form containing one or more active pharmaceutical ingredients and suitable excipients. Key characteristics include:
1. Composition: Mixture of active substances and excipients, typically in powder form
2. Manufacturing: Prepared by compression or molding of powders into a solid unit
3. Excipients may include:
   Diluents
   Binders or granulating agents
   Glidants (flow aids)
   Lubricants
   Disintegrants
   Sweeteners or flavors
   Pigments
4. Optional polymer coating for:
   Improved swallowability
   Controlled release of active ingredients
   Enhanced stability and shelf life
   Aesthetic purposes
5. Physical characteristics:
   Various shapes and colors for differentiation
   Often stamped with identifying symbols, letters, or numbers
   Sizes typically range from a few millimeters to about a centimeter
   May be scored or unscored

In the present context, the term “score” refers to a debossed line that runs across the planar surface of a solid oral dosage form (e.g., tablet), to facilitate splitting by breaking or cutting into smaller portions. This characteristic can be useful because the score can be used to facilitate the splitting of the tablet into fractions when less than a full tablet is desired for a dose. The fractions can be, e.g., of substantially equal size, such that relatively equal amounts of each of the two active ingredients can be administered over the divided doses. For example, with a scored oral tablet containing 125 mg nefazodone and 0.25 mg risperidone, each of the two fractions of the tablet will contain about 62.5 mg nefazodone and about 0.125 mg risperidone. Likewise, with a scored oral tablet containing 225 mg nefazodone and 0.50 mg risperidone, each of the two fractions of the tablet will contain about 112.5 mg nefazodone and about 0.25 mg risperidone. Additionally, with a scored oral tablet containing 275 mg nefazodone and 0.75 mg risperidone, each of the two fractions of the tablet will contain about 137.5 mg nefazodone and about 0.375 mg risperidone.

In the present context, the term “excipient” refers to an inactive substance (i.e., other than the active pharmaceutical ingredient(s)) used in the formulation of pharmaceutical product to bring functionality to the formulation. Suitable pharmaceutical excipients are described in, e.g., Handbook of Pharmaceutical Excipients, 9^{th} Edition, edited by Paul J Sheskey, Bruno C Hancock, Gary P Moss, David J Goldfarb (2020). The desired function of an excipient is to guarantee the required biopharmaceutical and physicochemical properties of the pharmaceutical product. Also, excipients for tablets are known as auxiliary substances. According to British Pharmacopoeia (BP), “Excipient is any constituent of a medicinal product that is not an active substance. Adjuvants, stabilizers, antimicrobial preservatives, diluents, antioxidants are excipients.” The ideal excipients will have the following characteristics: (1) An excipient will be physiologically inert; (2) Physically and chemically stable by themselves and in combination with active ingredient(s) or other excipients in a formulation; (3) Commercially available in an acceptable chemical and physical grade; (4) Compatible with active ingredient(s); (5) Nontoxic and acceptable by FDA or regulatory agencies; (6) It should have accepted organoleptic properties such as colorless or white to off-white color, odorless; (7) Economical (acceptably low); (8) Free from any impurities and microbial hazards; (9) They may not be contraindicated among them; (10) Do not hamper the bioavailability of active ingredients. Within the context of tablets, the below excipients are described.

In the present context, the term “diluents,” “fillers,” or “bulking agents” refers to excipients for tablets to increase the weight or volume. Diluents are fillers designed to make up the required bulk of the tablet when the drug dosage itself is inadequate to produce this bulk. Diluents are also known as fillers or bulking agents. Diluents provide improved cohesion, improve flow, allow direct compression manufacturing, and adjust tablet thickness or weight. Exemplary diluents used for solid oral dosages (e.g., tablets) include, e.g., microcrystalline cellulose; powdered cellulose [5-40% for wet granulation and 10-30% for dry granulation]; anhydrous lactose; lactose monohydrate; spray-dried lactose; mannitol; starch; pregelatinized starch; maize starch; corn starch; sorbitol; sucrose; compressible sugar (20-60%); confectioner's sugar (10-50%); sugar spheres; dextrates; dextrin; dextrose; calcium phosphate, dibasic, anhydrous; calcium carbonate; maltose; maltodextrin; kaolin; calcium phosphate, dibasic, dihydrate; tribasic calcium phosphate; calcium sulfate; cellaburate; calcium lactate; cellulose acetate; silicified microcrystalline cellulose; cellulose acetate; corn syrup; pregelatinized starch and corn starch; corn syrup solids; erythritol (30.0-90.0%); ethylcellulose (1.0-3.0%); ethyl acrylate and methyl methacrylate copolymer dispersion; fructose; isomalt; alpha-lactalbumin; lactitol; magnesium carbonate (direct compression ≤45); magnesium oxide; methacrylic acid and ethyl acrylate copolymer; methacrylic acid and methyl methacrylate copolymer; polydextrose; simethicone; pregelatinized modified starch; starch, pea; hydroxypropyl pea starch; starch, pregelatinized hydroxypropyl pea; potato starch; starch, hydroxypropyl potato; pregelatinized hydroxypropyl potato starch; starch, tapioca; wheat starch; starch hydrolysate, hydrogenated; pullulan; talc (5.0-30.0%); amino methacrylate copolymer; trehalose; and xylitol.

In the present context, the term “binder” or “binding agent” refers to excipients for tablets to facilitate the agglomeration of a powder into granules. According to USP, tablet binders are substances that are incorporated into formulations to facilitate the agglomeration of powder into granules during mixing with a granulating fluid such as water, hydroalcoholic mixtures, or other solvents. Tablet binders or binding agents are the substances that are added either dry or in liquid form during wet granulation to form granules or to promote cohesive compacts for directly compressed tablets. Binders are agents used to impart cohesive qualities to the powdered material. They impart cohesiveness to the tablet formulation that ensures the tablet remaining intact after compression, as well as improving the free-flowing qualities by the formulation of granules of desired hardness and size. Exemplary binders used for solid oral dosages (e.g., tablets) include, e.g., polyvinylpyrrolidone (also known as povidone); copovidone (2.0-5.0% in direct compression and 2.0-5.0% in wet granulation); carbomer (0.75-3.0%); corn starch and pregelatinized starch; pregelatinized starch (5-10%); carboxymethylcellulose sodium, carmellose sodium (1.0-6.0%); hypromellose/hydroxypropyl methylcellulose (HPMC), methocel (2-5%); PEG (polyethylene glycol); hydroxyethyl cellulose; hydroxypropyl cellulose (2.0-6.0%); hydroxyethylmethyl cellulose; calcium carboxymethylcellulose/calcium cellulose glycolate/carmellosum calcium (5-15%); guar galactomannan/guar gum (up to 10.0%); ethylcellulose; chitosan hydrochloride; dextrin; low-substituted hydroxypropyl cellulose; hydroxypropyl starch; ceratonia (0.15-0.75%); inulin; magnesium aluminum silicate (2.0-10.0%); maltodextrin (2-40% for direct compression and 3-10% for wet granulation); methylcellulose (1.0-5.0%); dextrates; polyethylene oxide (5.0-85.0%); povidone (0.5-5.0%); sodium alginate (1.0-3.0%); starch (3-20% w/w usually 5-10%); liquid glucose (5.0-10.0%); sucrose (2-20% dry granulation and 50-67% wet granulation); compressible sugar (5-20% as a dry binder in tablet formulations); zein (30% for wet granulation); gelatin (1-3% for wet mix); polymethacrylates (10-35% for dry mix and 15-35% as a solution and 4.5-10.5% w/w solids); sorbitol (2-10% for wet mix); glucose (2-25% for wet mix); sodium alginate (1-3% for wet mix); zein; and acacia (1.0-5.0%).

In the present context, the term “disintegrant” refers to excipients for tablets to assist dosage form's breakup or disintegration into small units/fragments. A disintegrant is a substance or a mixture of substances added to a tablet to facilitate its breakup or disintegration into small units/fragments and allow a drug substance to fast dissolution. According to USP, disintegrants are functional components that are added to formulations to promote rapid disintegration into smaller units and to allow a drug substance to dissolve more rapidly. When disintegrants come in contact with water or stomach or intestinal fluid, they absorb liquid and start to swell, dissolve, or form gels. This causes the tablet structure to rupture and disintegrate, making increased surfaces for improved dissolution of the drug substance. Exemplary disintegrants used for solid oral dosages (e.g., tablets) include, e.g., crospovidone (commercial name-kollidon cl) (2-5%); croscarmellose sodium (commercial name ac-di-sol, primellose) (10-25% in capsules and 0.5-5.0% in tablets. 2% w/w is used in direct compressed tablets and 3% w/w in wet-granulation processed tablets.); low-substituted hydroxypropyl cellulose; sodium starch glycolate (commercial name primogel, explotab) (2-8%, optimum concentration is about 4%, although 2% is sufficient in many cases); chitosan hydrochloride; corn starch and pregelatinized starch; calcium alginate & calcium sodium alginate (<10%); docusate sodium (≈0.5%); microcrystalline cellulose (5-15%); hydroxypropyl starch; magnesium aluminum silicate (2-10%); methylcellulose (2.0-10.0%); sodium alginate (2.5-10%); starch (3-25% w/w); pregelatinized starch (5-10%); calcium carboxymethylcellulose/calcium cellulose glycolate/carmellosum calcium (1-15%); and powdered cellulose (5-20%).

In the present context, the term “lubricant” refers to excipients for tablets to reduce the frictional forces between particle-particle as well as particles and metal-contact surfaces. Lubricants are non-toxic, pharmacologically inactive substances added to the formulation to prevent adhesion of the tablet material to the surface of the dies and punches, reducing interparticle friction, facilitating the ejection of the tablets from the die cavity, and improving the rate of flow of the tablet granulation. According to USP, lubricants are substances that typically are used to reduce the frictional forces between particles and between particles and metal-contact surfaces of manufacturing equipment such as tablet punches and dies used in the manufacture of solid dosage forms. Before compaction, liquid lubricants may be absorbed into the tablet granule matrix. Exemplary lubricants used for solid oral dosages (e.g., tablets) include, e.g., magnesium stearate; magnesium silicate; calcium stearate; sodium lauryl sulphate; sodium stearyl fumarate; magnesium lauryl sulphate; stearic acid; calcium stearate; glyceryl behenate; behenoyl polyoxylglycerides; glyceryl dibehenate; lauric acid; glyceryl monostearate; glyceryl tristearate; myristic acid; palmitic acid; poloxamer; polyethylene glycol; polyethylene glycol 3350; polysorbate 20; polyoxyl 10 oleyl ether; polyoxyl 15 hydroxystearate; polysorbate 40; polyoxyl 20 cetostearyl ether; polyoxyl 40 stearate; polysorbate 60; polysorbate 80; potassium benzoate; sodium benzoate; sorbitan monolaurate; sorbitan monooleate; sodium stearate; sorbitan monopalmitate; sorbitan monostearate; zinc stearate; sorbitan sesquioleate; sorbitan trioleate; and talc.

In the present context, the term “glidant” and “anticaking agent” refers to excipients used to promote the flow properties of tablet granules or power materials. Glidants are non-toxic, pharmacologically inactive substance used to promote the flow properties of tablet granulation or powder materials by decreasing interparticle friction and cohesion. These always are added in the dry state during the lubrication step before compression. According to USP, glidants and anticaking agents are used to promote powder flow and to reduce the caking or clumping that can occur when powders are stored in bulk. Additionally, glidants and anticaking agents reduce the incidence of bridging during the emptying of powder hoppers and powder processing. Exemplary glidants used for solid oral dosages (e.g., tablets) include, e.g., colloidal silicon dioxide (trade name: aerosil 200/cab-o-sil); talc; tribasic calcium phosphate; calcium silicate; cellulose, powdered; magnesium oxide; sodium stearate; magnesium silicate; silica, dental-type; magnesium trisilicate; and hydrophobic colloidal silica.

In the present context, the term “coloring agents” or “colorant” refers to excipients used to give a color or identification of the tablets as either pigment or coating materials. Coloring agents are inactive substance(s) added into dosage forms to produce a distinctive appearance that may serve to differentiate a product from others that have a similar physical appearance or in some instances, to protect photolabile components of the dosage form. Coloring agents are categorized into: (1) Dyes: water-soluble coloring substances, (2) Lakes: insoluble forms of a dye that result from its irreversible adsorption onto a hydrous metal oxide, (3) Inorganic pigments: substances such as titanium dioxide or iron oxides, and (4) Natural colorants: colored compounds not considered dyes, such as riboflavin. Exemplary coloring agents used for solid oral dosages (e.g., tablets) include, e.g., caramel; ferric oxide; titanium dioxide; ferrosoferric oxide; aluminum oxide; FD & C red #40/allura red AC; amaranth; FD & C blue #1/brilliant blue FCF; canthaxanthin; carmine; carmoisine (azorubine); curcumin (tumeric); FD & C red #3/erythrosine; fast green FCF; green S (lissamine green); D & C red #30/helendon pink; FD & C blue #2/indigo carmine; iron oxide black; iron oxide red; D & C red #7/lithol rubin BK; patent blue V; D & C red #28/phloxine B; iron oxide yellow; D & C red #27/phloxine 0; ponceau 4R (cochineal red A); quinoline yellow WS; D & C yellow #10; riboflavin (lactoflavin); FD & C yellow #5/tartrazine; and FD & C yellow #6/sunset yellow FCF.

In the present context, the term “flavoring agent” refers to excipients used in some types of tablets (e.g., chewable tablets or dispersible tablets) or in coating suspension to impart a pleasant flavor. According to USP, a flavor is a single chemical entity or a blend of chemicals of synthetic or natural origin that can produce a taste or aroma (i.e., fragrance) response when orally consumed or smelled. Flavoring agents are consumed orally and appreciated by both smell and taste while fragrances are only for external use and appreciated only by smell. Generally, flavors mask the unpleasant smell as well as taste and to make the product more palatable, thus increasing patient compliance. Exemplary flavoring agents used for solid oral dosages (e.g., tablets) include, e.g., vanillin; peppermint flavor powder; berry flavor powder; strawberry flavor powder; orange flavor powder; lemon flavor powder; orange essence; ethyl maltol; eucalyptus oil; isobutyl alcohol; sodium succinate; adipic acid; almond oil; anethole; benzaldehyde; denatonium benzoate; ethyl acetate; ethyl vanillin; ethylcellulose; fructose; fumaric acid; 1-glutamic acid, hydrochloride; lactitol; leucine; malic acid; maltol; menthol/racementhol; methionine; methyl salicylate; monosodium glutamate; peppermint oil; strawberry flavor; peppermint spirit; racemethionine; rose oil; rose water; sodium acetate; sodium lactate solution; tartaric acid; thymol; fumaric acid; inulin; isomalt; and neohesperidin dihydrochalcone.

In the present context, the term “sweetener” or “sweetening agent” refers to excipients used in some types of tablets (e.g., chewable tablets or dispersible tablets) or in coating suspension to impart a sweet flavor. Sweeteners are substances used to mask the unpleasant taste and sweeten oral dosage forms and also to mask unpleasant flavors. It binds to receptors on the tongue that are responsible for the sensation of sweetness. Sucrose is the standard for sweetness. Exemplary sweeteners used for solid oral dosages (e.g., tablets) include, e.g., sucralose; saccharin sodium; neotame; sucrose; acesulfame potassium; aspartame; aspartame acesulfame; corn syrup; corn syrup solids; dextrates; dextrose; dextrose excipient; erythritol; fructose; galactose; glucose; glycerin; inulin; invert sugar; isomalt; lactitol; maltitol; maltose; mannitol; saccharin; saccharin calcium; sorbitol; starch hydrolysate, hydrogenated; sugar, compressible; sugar, confectioner's; tagatose; trehalose; and xylitol.

In the present context, the term “surfactant” refers to excipients used for low solubility tablets to improve wetting and deagregation of drug particles to get a rapid and improved dissolution. Surfactants are substances with well-defined polar and non-polar regions that allow them to aggregate in solution to form micelles and non-polar drugs can partition into these micelles and be solubilized. They may decrease the surface tension (or interfacial tension) between a liquid and a solid or between a gas and a liquid or two liquids. Exemplary surfactants used for solid oral dosages (e.g., tablets) include, e.g., behenoyl polyoxylglycerides; polysorbate 20; polysorbate 40; docusate sodium; polysorbate 60; polysorbate 80; benzalkonium chloride; caprylocaproyl polyoxylglycerides; cetylpyridinium chloride; lauroyl polyoxylglycerides; linoleoyl polyoxylglycerides; octoxynol 9; oleoyl polyoxylglycerides; poloxamer; polyoxyl 10 oleyl ether; polyoxyl 15 hydroxystearate; nonoxynol 9; polyoxyl 20 cetostearyl ether; polyoxyl 40 stearate; pullulan; polyoxyl lauryl ether; polyoxyl stearyl ether; sodium lauryl sulfate; sorbitan monolaurate; sorbitan monooleate; polyoxyl stearate; sorbitan monopalmitate; sorbitan monostearate; stearoyl polyoxylglycerides; sorbitan sesquioleate; sorbitan trioleate; and tyloxapol.

In the present context, the term “coating material” refers to excipients used as a film former to facilitate ease in swallowing. These are substance used to coat tablets or particles. Coating materials are excipients for tablets, but not for all tablets. A proper coating formulation includes the following materials: film formers (which may be enteric or non-enteric); solvents; plasticizers; colorants; opaquant-extenders; and miscellaneous coating solution components.

### Specific Ranges, Values, and Embodiments

The specific embodiments provided below are for illustration purposes only, and do not otherwise limit the scope of the disclosed subject matter, as defined by the claims.

### Methods of Medical Treatment

In specific embodiments, a unit dose containing nefazodone hydrochloride, risperidone, and one or more pharmaceutically acceptable excipients is administered to a person.

In specific embodiments, a unit dose containing nefazodone hydrochloride, risperidone, and one or more pharmaceutically acceptable excipients is administered to a person suffering from a psychiatric disorder or mental disorder.

In specific embodiments, a unit dose containing nefazodone hydrochloride, risperidone, and one or more pharmaceutically acceptable excipients is administered to a person suffering from a psychiatric disorder that includes suicidal behavior, suicidal ideation, and/or major depressive disorder (MDD).

In specific embodiments, a unit dose containing nefazodone hydrochloride, risperidone, and one or more pharmaceutically acceptable excipients is administered to a person suffering from suicidal behavior.

In specific embodiments, a unit dose containing nefazodone hydrochloride, risperidone, and one or more pharmaceutically acceptable excipients is administered to a person suffering from at least one of the subtypes of suicidal behavior: (i) suicide attempt, (ii) interrupted attempt, (iii) aborted attempt, and (iv) preparatory actions toward imminent suicidal behaviors.

In specific embodiments, a unit dose containing nefazodone hydrochloride, risperidone, and one or more pharmaceutically acceptable excipients is administered to a person suffering from the subtype of suicidal behavior, suicide attempt.

In specific embodiments, a unit dose containing nefazodone hydrochloride, risperidone, and one or more pharmaceutically acceptable excipients is administered to a person suffering from the subtype of suicidal behavior, interrupted attempt.

In specific embodiments, a unit dose containing nefazodone hydrochloride, risperidone, and one or more pharmaceutically acceptable excipients is administered to a person suffering from the subtype of suicidal behavior, aborted attempt.

In specific embodiments, a unit dose containing nefazodone hydrochloride, risperidone, and one or more pharmaceutically acceptable excipients is administered to a person suffering from the subtype of suicidal behavior, preparatory actions toward imminent suicidal behaviors.

In specific embodiments, a unit dose containing nefazodone hydrochloride, risperidone, and one or more pharmaceutically acceptable excipients is administered to a person suffering from suicidal ideation.

In specific embodiments, a unit dose containing nefazodone hydrochloride, risperidone, and one or more pharmaceutically acceptable excipients is administered to a person suffering from chronic suicidal ideation.

In specific embodiments, a unit dose containing nefazodone hydrochloride, risperidone, and one or more pharmaceutically acceptable excipients is administered to a person suffering from at least one of the subtypes of chronic suicidal ideation: (i) passive, (ii) active: nonspecific (no method, intent, or plan), (iii) active: method, but no intent or plan, (iv) active: method and intent, but no plan, and (v) active: method, intent, and plan.

In specific embodiments, a unit dose containing nefazodone hydrochloride, risperidone, and one or more pharmaceutically acceptable excipients is administered to a person suffering from the subtype of chronic suicidal ideation, passive.

In specific embodiments, a unit dose containing nefazodone hydrochloride, risperidone, and one or more pharmaceutically acceptable excipients is administered to a person suffering from the subtype of chronic suicidal ideation, active: nonspecific (no method, intent, or plan).

In specific embodiments, a unit dose containing nefazodone hydrochloride, risperidone, and one or more pharmaceutically acceptable excipients is administered to a person suffering from the subtype of chronic suicidal ideation, active: method, but no intent or plan.

In specific embodiments, a unit dose containing nefazodone hydrochloride, risperidone, and one or more pharmaceutically acceptable excipients is administered to a person suffering from the subtype of chronic suicidal ideation, active: method and intent, but no plan.

In specific embodiments, a unit dose containing nefazodone hydrochloride, risperidone, and one or more pharmaceutically acceptable excipients is administered to a person suffering from the subtype of chronic suicidal ideation, active: method, intent, and plan.

In specific embodiments, a unit dose containing nefazodone hydrochloride, risperidone, and one or more pharmaceutically acceptable excipients is administered to a person suffering from major depressive disorder (MDD).

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons at risk of committing suicide, persons having recently attempted to commit suicide, and/or persons having a history of suicide attempts.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons at risk of committing suicide.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons having recently attempted to commit suicide.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons having a history of suicide attempts.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons that are afflicted with treatment-refractory depression.

In specific embodiments, the person having suicidal thoughts and/or suicidal behavior suffers from depression.

In specific embodiments, the person having suicidal thoughts and/or suicidal behavior suffers from depression, including major or severe depression, depression with anxiety symptoms in the form of either anxiety disorders as defined in DSM-5 (Diagnostic and Statistical manual of Mental Disorders, Fifth Edition) or associated anxiety symptoms and in connection with other disorders involving depression.

In specific embodiments, the person having suicidal thoughts and/or suicidal behavior suffers from major or severe depression.

In specific embodiments, the person having suicidal thoughts and/or suicidal behavior suffers from major depressive disorder (MDD).

In specific embodiments, the person having suicidal thoughts and/or suicidal behavior suffers from a psychotic illness.

In specific embodiments, the person having suicidal thoughts and/or suicidal behavior suffers from a psychotic illness, such as schizophrenia or schizoaffective psychosis.

In specific embodiments, the person having suicidal thoughts and/or suicidal behavior suffers from depression and has previously undergone treatment with an antidepressant and/or an antipsychotic.

In specific embodiments, a unit dose containing nefazodone hydrochloride, risperidone, and one or more pharmaceutically acceptable excipients is administered to a person for the treatment, mitigation, and/or reduction of suicidal thoughts and/or suicidal behavior.

In specific embodiments, the person having suicidal thoughts and/or suicidal behavior fulfils the following inclusion criteria: (a) DSM-5 (i) major depression with symptoms present for a minimum of 4 weeks; (b) minimum total score of 22 on the 10-item MADRS (Montgomery SA, Asberg M., A new depression scale designed to be sensitive to change., Br. J. Psychiatry 1979, 134, 382-389) and a score of 2 or more on the HAM-D item (depressed mood) (Hamilton, M., Br. J. Soc. Clin. Psychol 1967, 6, 278-296). Persons who still fulfilled the MADRS and HAM-D criteria after 1-week, single-blind placebo lead-in were randomized to the double-blind treatment.

In specific embodiments, the person having suicidal thoughts and/or suicidal behavior fulfils the following inclusion criteria: (i) Subject meets Diagnostic and Statistical Manual of Mental Disorders (5th edition) (DSM-5) diagnostic criteria for a MDE, based upon clinical assessment and confirmed by the Mini International Psychiatric Interview (MINI); (ii) Subjects has chronic suicidal ideation, confirmed by a score of ≥3 (at least moderate) in the CGI-SS-r for at least 6 weeks (a suicidal ideation for most of the day, for more days than not) prior to screening; (iii) subject has a MADRS total score of ≥24 at Screening and Baseline; (iv) Subject is receiving a therapeutic dose of an antidepressant treatment (ADT) with or without augmentation for at least 8 weeks prior to screening; and (v) Subject can, in the opinion of the investigator, be safely managed as an outpatient for the entire treatment.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that meets the Diagnostic and Statistical Manual of Mental Disorders (5th edition) (DSM-5) diagnostic criteria for a MDE, based upon clinical assessment and confirmed by the Mini International Psychiatric Interview (MINI).

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that has chronic suicidal ideation, confirmed by a score of ≥3 (at least moderate) in the CGI-SS-r for at least 6 weeks (a suicidal ideation for most of the day, for more days than not) prior to screening.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that has a MADRS total score of ≥24 at Screening and Baseline.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that is receiving a therapeutic dose of an antidepressant treatment (ADT) with or without augmentation for at least 8 weeks prior to screening.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that can, in the opinion of the investigator, be safely managed as an outpatient for the entire treatment.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥1 (questionably suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥2 (mildly suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥3 (moderately suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥4 (markedly suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥5 (severely suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 6 (among the most extremely suicidal patients).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 1-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 2-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 3-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 4-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 5-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 6.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤5.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤4.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤3.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤2.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥1 unit in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥2 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥3 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥4 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of 5 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement to a score of 0 or 1 in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement to a score of 0 in the Global Impression of Severity of Suicidality-revised (CGI-SS-r) evaluation score.

In specific embodiments, the pharmaceutical product is a maintenance drug, prescribed to treat chronic, long-term psychiatric conditions and is taken on a regular, recurring, and ongoing basis. In those embodiments, the administration is long-term and continuous.

In specific embodiments, the administration is a twice-a-day (BID) administration.

In specific embodiments, the administration is a twice-a-day (BID) administration, once in the morning and once in the evening.

In specific embodiments, the method of treating a person suffering from a psychiatric disorder continues, provided the psychiatric disorder does not worsen.

In specific embodiments, the method of treating a person suffering from a psychiatric disorder continues, provided the severity of the psychiatric disorder remains about the same.

In specific embodiments, the method of treating a person suffering from a psychiatric disorder continues, provided the psychiatric disorder improves.

In specific embodiments, the method of treating a person suffering from a psychiatric disorder continues for a period of time effective to treat the psychiatric disorder, provided (i) the psychiatric disorder does not worsen, or (ii) the severity of the psychiatric disorder remains about the same, or (iii) the psychiatric disorder improves.

### Nefazodone Hydrochloride

In specific embodiments, the unit dose contains nefazodone (or a pharmaceutically acceptable salt thereof), risperidone, and one or more pharmaceutically acceptable excipients.

In specific embodiments, the nefazodone is present in the unit dose as the free base.

In specific embodiments, the nefazodone is present in the unit dose as a pharmaceutically acceptable salt thereof.

In specific embodiments, the nefazodone is present in the unit dose as the hydrochloride salt.

In specific embodiments, the unit dose includes nefazodone hydrochloride in 12.5-325 mg.

In specific embodiments, the unit dose includes nefazodone hydrochloride in 25-200 mg.

In specific embodiments, the unit dose includes nefazodone hydrochloride in 25±15 mg.

In specific embodiments, the unit dose includes nefazodone hydrochloride in 25±5 mg.

In specific embodiments, the unit dose includes nefazodone hydrochloride in 25 mg.

In specific embodiments, the unit dose includes nefazodone hydrochloride in 50±25 mg.

In specific embodiments, the unit dose includes nefazodone hydrochloride in 50±5 mg.

In specific embodiments, the unit dose includes nefazodone hydrochloride in 50 mg.

In specific embodiments, the unit dose includes nefazodone hydrochloride in 75±25 mg.

In specific embodiments, the unit dose includes nefazodone hydrochloride in 75±5 mg.

In specific embodiments, the unit dose includes nefazodone hydrochloride in 75 mg.

In specific embodiments, the unit dose includes nefazodone hydrochloride in 125±25 mg.

In specific embodiments, the unit dose includes nefazodone hydrochloride in 125±15 mg.

In specific embodiments, the unit dose includes nefazodone hydrochloride in 125 mg.

In specific embodiments, the unit dose includes nefazodone hydrochloride in 175±25 mg.

In specific embodiments, the unit dose includes nefazodone hydrochloride in 175±15 mg.

In specific embodiments, the unit dose includes nefazodone hydrochloride in 175 mg.

In specific embodiments, the unit dose includes nefazodone hydrochloride in 200±25 mg.

In specific embodiments, the unit dose includes nefazodone hydrochloride in 200±15 mg.

In specific embodiments, the unit dose includes nefazodone hydrochloride in 200 mg.

### Daily Dosing

In specific embodiments, the nefazodone hydrochloride is administered in 50±25 mg per day.

In specific embodiments, the nefazodone hydrochloride is administered in 50±10 mg per day.

In specific embodiments, the nefazodone hydrochloride is administered in 50±5 mg per day.

In specific embodiments, the nefazodone hydrochloride is administered in 50 mg per day.

In specific embodiments, the nefazodone hydrochloride is administered in 100±50 mg per day.

In specific embodiments, the nefazodone hydrochloride is administered in 100±25 mg per day.

In specific embodiments, the nefazodone hydrochloride is administered in 100±10 mg per day.

In specific embodiments, the nefazodone hydrochloride is administered in 100±5 mg per day.

In specific embodiments, the nefazodone hydrochloride is administered in 100 mg per day.

In specific embodiments, the nefazodone hydrochloride is administered in 150±50 mg per day.

In specific embodiments, the nefazodone hydrochloride is administered in 150±25 mg per day.

In specific embodiments, the nefazodone hydrochloride is administered in 150±10 mg per day.

In specific embodiments, the nefazodone hydrochloride is administered in 150±5 mg per day.

In specific embodiments, the nefazodone hydrochloride is administered in 150 mg per day.

In specific embodiments, the nefazodone hydrochloride is administered in 250±50 mg per day.

In specific embodiments, the nefazodone hydrochloride is administered in 250±25 mg per day.

In specific embodiments, the nefazodone hydrochloride is administered in 250±10 mg per day.

In specific embodiments, the nefazodone hydrochloride is administered in 250±5 mg per day.

In specific embodiments, the nefazodone hydrochloride is administered in 250 mg per day.

In specific embodiments, the nefazodone hydrochloride is administered in 350±50 mg per day.

In specific embodiments, the nefazodone hydrochloride is administered in 350±25 mg per day.

In specific embodiments, the nefazodone hydrochloride is administered in 350±10 mg per day.

In specific embodiments, the nefazodone hydrochloride is administered in 350±5 mg per day.

In specific embodiments, the nefazodone hydrochloride is administered in 350 mg per day.

In specific embodiments, the nefazodone hydrochloride is administered in 400±50 mg per day.

In specific embodiments, the nefazodone hydrochloride is administered in 400±25 mg per day.

In specific embodiments, the nefazodone hydrochloride is administered in 400±10 mg per day.

In specific embodiments, the nefazodone hydrochloride is administered in 400±5 mg per day.

In specific embodiments, the nefazodone hydrochloride is administered in 400 mg per day.

### Risperidone

In specific embodiments, the unit dose contains nefazodone (or a pharmaceutically acceptable salt thereof), risperidone, and one or more pharmaceutically acceptable excipients.

In specific embodiments, the unit dose includes risperidone in 0.1-1.0 mg.

In specific embodiments, the unit dose includes risperidone in 0.125-0.75 mg.

In specific embodiments, the unit dose includes risperidone in 0.125±0.05 mg.

In specific embodiments, the unit dose includes risperidone in 0.125±0.025 mg.

In specific embodiments, the unit dose includes risperidone in 0.125±0.01 mg.

In specific embodiments, the unit dose includes risperidone in 0.125 mg.

In specific embodiments, the unit dose includes risperidone in 0.25±0.15 mg.

In specific embodiments, the unit dose includes risperidone in 0.25±0.10 mg.

In specific embodiments, the unit dose includes risperidone in 0.25±0.05 mg.

In specific embodiments, the unit dose includes risperidone in 0.25 mg.

In specific embodiments, the unit dose includes risperidone in 0.375±0.15 mg.

In specific embodiments, the unit dose includes risperidone in 0.375±0.10 mg.

In specific embodiments, the unit dose includes risperidone in 0.375±0.05 mg.

In specific embodiments, the unit dose includes risperidone in 0.375 mg.

In specific embodiments, the unit dose includes risperidone in 0.5±0.25 mg.

In specific embodiments, the unit dose includes risperidone in 0.5±0.15 mg.

In specific embodiments, the unit dose includes risperidone in 0.5±0.10 mg.

In specific embodiments, the unit dose includes risperidone in 0.5±0.05 mg.

In specific embodiments, the unit dose includes risperidone in 0.5 mg.

In specific embodiments, the unit dose includes risperidone in 0.625±0.20 mg.

In specific embodiments, the unit dose includes risperidone in 0.625±0.15 mg.

In specific embodiments, the unit dose includes risperidone in 0.625±0.10 mg.

In specific embodiments, the unit dose includes risperidone in 0.625±0.05 mg.

In specific embodiments, the unit dose includes risperidone in 0.625 mg.

In specific embodiments, the unit dose includes risperidone in 0.75±0.25 mg.

In specific embodiments, the unit dose includes risperidone in 0.75±0.15 mg.

In specific embodiments, the unit dose includes risperidone in 0.75±0.10 mg.

In specific embodiments, the unit dose includes risperidone in 0.75±0.05 mg.

In specific embodiments, the unit dose includes risperidone in 0.75 mg.

### Daily Dosing

In specific embodiments, the risperidone is administered in 0.1-2.0 mg per day.

In specific embodiments, the risperidone is administered in 0.25-1.5 mg per day.

In specific embodiments, the risperidone is administered in 0.25±0.10 mg per day.

In specific embodiments, the risperidone is administered in 0.25±0.05 mg per day.

In specific embodiments, the risperidone is administered in 0.25±0.025 mg per day.

In specific embodiments, the risperidone is administered in 0.25±0.01 mg per day.

In specific embodiments, the risperidone is administered in 0.25 mg per day.

In specific embodiments, the risperidone is administered in 0.5±0.25 mg per day.

In specific embodiments, the risperidone is administered in 0.5±0.10 mg per day.

In specific embodiments, the risperidone is administered in 0.5±0.05 mg per day.

In specific embodiments, the risperidone is administered in 0.5±0.025 mg per day.

In specific embodiments, the risperidone is administered in 0.5 mg per day.

In specific embodiments, the risperidone is administered in 0.75±0.25 mg per day.

In specific embodiments, the risperidone is administered in 0.75±0.10 mg per day.

In specific embodiments, the risperidone is administered in 0.75±0.05 mg per day.

In specific embodiments, the risperidone is administered in 0.75±0.025 mg per day.

In specific embodiments, the risperidone is administered in 0.75 mg per day.

In specific embodiments, the risperidone is administered in 1.0±0.25 mg per day.

In specific embodiments, the risperidone is administered in 1.0±0.10 mg per day.

In specific embodiments, the risperidone is administered in 1.0±0.05 mg per day.

In specific embodiments, the risperidone is administered in 1.0±0.025 mg per day.

In specific embodiments, the risperidone is administered in 1.0 mg per day.

In specific embodiments, the risperidone is administered in 1.25±0.5 mg per day.

In specific embodiments, the risperidone is administered in 1.25±0.25 mg per day.

In specific embodiments, the risperidone is administered in 1.25±0.10 mg per day.

In specific embodiments, the risperidone is administered in 1.25±0.05 mg per day.

In specific embodiments, the risperidone is administered in 1.25±0.025 mg per day.

In specific embodiments, the risperidone is administered in 1.25 mg per day.

In specific embodiments, the risperidone is administered in 1.5±0.5 mg per day.

In specific embodiments, the risperidone is administered in 1.5±0.25 mg per day.

In specific embodiments, the risperidone is administered in 1.5±0.10 mg per day.

In specific embodiments, the risperidone is administered in 1.5±0.05 mg per day.

In specific embodiments, the risperidone is administered in 1.5±0.025 mg per day.

In specific embodiments, the risperidone is administered in 1.5 mg per day.

### Combination

The unit dose can include risperidone and nefazodone hydrochloride in the amounts shown below, within the dosing regimen.

| | |
|---|---|
| | Nefazodone |
| Risperidone | hydrochloride |

Initial Dose:

| | | | | |
|---|---|---|---|---|
| | 0.1875 ± 0.065 | mg | 25 ± 5 | mg |
| Option A | 0.125 | mg | 25 | mg |
| Option B | 0.25 | mg | 25 | mg |

Titrated Dose:

| | | | | |
|---|---|---|---|---|
| | 0.55 ± 0.3 | mg | 125 ± 75 | mg |
| Option A-dose 1 | 0.375 | mg | 75 | mg |
| Option B-dose 1 | 0.5 | mg | 75 | mg |
| Option A-dose 2 | 0.625 | mg | 125 | mg |
| Option B-dose 2 | 0.75 | mg | 125 | mg |
| Option A-dose 3 | 0.75 | mg | 175 | mg |
| Option B-dose 3 | 0.75 | mg | 175 | mg |
| Option A-dose 4 | 0.75 | mg | 200 | mg |
| Option B-dose 4 | 0.75 | mg | 200 | mg |

Maintenance Dose:

| | | | | |
|---|---|---|---|---|
| | ≥0.75 | mg | ≥200 | mg |
| | 0.75-1.0 | mg | 200-300 | mg |
| Option A/B | 0.75 | mg | 200 | mg |

The risperidone and nefazodone hydrochloride can be administered in the daily amounts shown below, within the dosing regimen.

| | |
|---|---|
| | Nefazodone |
| Risperidone | hydrochloride |

Initial Dose:

| | | | | |
|---|---|---|---|---|
| | 0.375 ± 0.2 | mg | 50 ± 10 | mg |
| Option A | 0.25 | mg | 50 | mg |
| Option B | 0.50 | mg | 50 | mg |

Titrated Dose:

| | | | | |
|---|---|---|---|---|
| | 1.125 ± 0.5 | mg | 275 ± 200 | mg |
| Option A-dose 1 | 0.75 | mg | 150 | mg |
| Option B-dose 1 | 1.0 | mg | 150 | mg |
| Option A-dose 2 | 1.25 | mg | 250 | mg |
| Option B-dose 2 | 1.5 | mg | 250 | mg |
| Option A-dose 3 | 1.5 | mg | 350 | mg |
| Option B-dose 3 | 1.5 | mg | 350 | mg |
| Option A-dose 4 | 1.5 | mg | 400 | mg |
| Option B-dose 4 | 1.5 | mg | 400 | mg |

Maintenance Dose:

| | | | | |
|---|---|---|---|---|
| | ≥1.5 | mg | ≥400 | mg |
| | 1.5-2.0 | mg | 400-600 | mg |
| Option A/B | 1.5 | mg | 400 | mg |

In specific embodiments, the initial dosing includes administering 0.375±0.2 mg per day risperidone and 50±10 mg per day nefazodone hydrochloride, in divided doses twice daily, for 1-4 weeks. Specifically, during the initial dosing, the daily amount of risperidone can remain the same, or can vary. Likewise, during the initial dosing, the daily amount of nefazodone hydrochloride can remain the same, or can vary. Either way, each day during the initial dosing, 0.375±0.2 mg per day risperidone and 50±10 mg per day nefazodone hydrochloride is administered, in divided doses, twice daily.

In more specific embodiments, the initial dosing includes administering 0.375±0.2 mg per day risperidone and 50±10 mg per day nefazodone hydrochloride, in divided doses twice daily, for 1-3 weeks.

In more specific embodiments, the initial dosing includes administering 0.375±0.2 mg per day risperidone and 50±10 mg per day nefazodone hydrochloride, in divided doses twice daily, for 1-2 weeks.

In more specific embodiments, the initial dosing includes administering 0.375±0.2 mg per day risperidone and 50±10 mg per day nefazodone hydrochloride, in divided doses twice daily, for 1 week.

In more specific embodiments, the initial dosing includes administering 0.375±0.15 mg per day risperidone and 50±7.5 mg per day nefazodone hydrochloride, in divided doses twice daily, for 1-2 weeks.

In more specific embodiments, the initial dosing includes administering 0.375±0.125 mg per day risperidone and 50±5 mg per day nefazodone hydrochloride, in divided doses twice daily, for 1-2 weeks.

The dosing escalation (titration) includes gradually increasing the dosage of both risperidone and nefazodone hydrochloride, over a period of time, from the initial dose to the maintenance dose. The dose of nefazodone hydrochloride is increased from the initial dosing of 50±10 mg per day, in increments of about 100±25 mg per day, at intervals of no less than 1 week, to the maintenance dose of 500±100 mg per day. Likewise, the dose of risperidone is increased from the initial dosing of 0.375±0.2 per day, in increments of about 0.50±0.25 mg per day, at intervals of no less than 1 week, to the maintenance dose of 1.75±0.5 mg per day.

In specific embodiments, the dosing escalation includes increasing the dose of nefazodone hydrochloride 100±25 mg per day, increased at intervals of no less than 1 week.

In specific embodiments, the dosing escalation includes increasing the dose of nefazodone hydrochloride 100±20 mg per day, increased at intervals of no less than 1 week.

In specific embodiments, the dosing escalation includes increasing the dose of nefazodone hydrochloride 100±15 mg per day, increased at intervals of no less than 1 week.

In specific embodiments, the dosing escalation includes increasing the dose of nefazodone hydrochloride 100±10 mg per day, increased at intervals of no less than 1 week.

In specific embodiments, the dosing escalation includes increasing the dose of nefazodone hydrochloride 100±5 mg per day, increased at intervals of no less than 1 week.

In specific embodiments, the dosing escalation includes increasing the dose of nefazodone hydrochloride 100 mg per day, increased at intervals of no less than 1 week.

In specific embodiments, the dosing escalation includes increasing the dose of nefazodone hydrochloride at intervals of no less than 1 week.

In specific embodiments, the dosing escalation includes increasing the dose of nefazodone hydrochloride at intervals of 1-2 weeks.

In specific embodiments, the dosing escalation includes increasing the dose of nefazodone hydrochloride at intervals of 1 week.

In specific embodiments, the dosing escalation includes increasing the dose of nefazodone hydrochloride over a period of time of ≤8 weeks.

In specific embodiments, the dosing escalation includes increasing the dose of nefazodone hydrochloride over a period of time of ≤7 weeks.

In specific embodiments, the dosing escalation includes increasing the dose of nefazodone hydrochloride over a period of time of ≤6 weeks.

In specific embodiments, the dosing escalation includes increasing the dose of nefazodone hydrochloride over a period of time of ≤5 weeks.

In specific embodiments, the dosing escalation includes increasing the dose of nefazodone hydrochloride over a period of time of ≤4 weeks.

In specific embodiments, the dosing escalation includes increasing the dose of nefazodone hydrochloride over a period of time of 1-4 weeks.

In specific embodiments, the dosing escalation includes increasing the dose of nefazodone hydrochloride over a period of time of 2-4 weeks.

In specific embodiments, the dosing escalation includes increasing the dose of nefazodone hydrochloride over a period of time of 2-3 weeks.

In specific embodiments, the dosing escalation includes increasing the dose of risperidone 0.50±0.25 mg per day mg per day, increased at intervals of no less than 1 week.

In specific embodiments, the dosing escalation includes increasing the dose of risperidone 0.50±0.2 mg per day mg per day, increased at intervals of no less than 1 week.

In specific embodiments, the dosing escalation includes increasing the dose of risperidone 0.50±0.15 mg per day mg per day, increased at intervals of no less than 1 week.

In specific embodiments, the dosing escalation includes increasing the dose of risperidone 0.50±0.10 mg per day mg per day, increased at intervals of no less than 1 week.

In specific embodiments, the dosing escalation includes increasing the dose of risperidone 0.50±0.05 mg per day mg per day, increased at intervals of no less than 1 week.

In specific embodiments, the dosing escalation includes increasing the dose of risperidone 0.50 mg per day mg per day, increased at intervals of no less than 1 week.

In specific embodiments, the dosing escalation includes increasing the dose of risperidone at intervals of no less than 1 week.

In specific embodiments, the dosing escalation includes increasing the dose of risperidone at intervals of 1-2 weeks.

In specific embodiments, the dosing escalation includes increasing the dose of risperidone at intervals of 1 week.

In specific embodiments, the dosing escalation includes increasing the dose of risperidone over a period of time of ≤8 weeks.

In specific embodiments, the dosing escalation includes increasing the dose of risperidone over a period of time of ≤7 weeks.

In specific embodiments, the dosing escalation includes increasing the dose of risperidone over a period of time of ≤6 weeks.

In specific embodiments, the dosing escalation includes increasing the dose of risperidone over a period of time of ≤5 weeks.

In specific embodiments, the dosing escalation includes increasing the dose of risperidone over a period of time of ≤4 weeks.

In specific embodiments, the dosing escalation includes increasing the dose of risperidone over a period of time of 1-4 weeks.

In specific embodiments, the dosing escalation includes increasing the dose of risperidone over a period of time of 2-4 weeks.

In specific embodiments, the dosing escalation includes increasing the dose of risperidone over a period of time of 2-3 weeks.

The maintenance dosing includes administering 500±150 mg per day nefazodone hydrochloride and 1.75±0.5 mg per day risperidone, in divided doses, twice daily. During the maintenance dosing, the dose of nefazodone hydrochloride and/or risperidone can optionally be optimized (e.g., adjusted). Specifically, during the maintenance dosing, the daily amount of risperidone can remain the same, or can vary. Likewise, during the maintenance dosing, the daily amount of nefazodone hydrochloride can remain the same, or can vary. Either way, each day during the maintenance dosing, 1.75±0.5 mg per day risperidone and 500±150 mg per day nefazodone hydrochloride is administered, in divided doses, twice daily.

In specific embodiments, the maintenance dosing includes administering 500±150 mg per day nefazodone hydrochloride, in divided doses, twice daily.

In specific embodiments, the maintenance dosing includes administering 500±125 mg per day nefazodone hydrochloride, in divided doses, twice daily.

In specific embodiments, the maintenance dosing includes administering 500±100 mg per day nefazodone hydrochloride, in divided doses, twice daily.

In specific embodiments, the maintenance dosing includes administering 400±50 mg per day nefazodone hydrochloride, in divided doses, twice daily.

In specific embodiments, the maintenance dosing includes administering 400±25 mg per day nefazodone hydrochloride, in divided doses, twice daily.

In specific embodiments, the maintenance dosing includes administering 400±15 mg per day nefazodone hydrochloride, in divided doses, twice daily.

In specific embodiments, the maintenance dosing includes administering 400±10 mg per day nefazodone hydrochloride, in divided doses, twice daily.

In specific embodiments, the maintenance dosing includes administering 400±5 mg per day nefazodone hydrochloride, in divided doses, twice daily.

In specific embodiments, the maintenance dosing includes administering 400 mg per day nefazodone hydrochloride, in divided doses, twice daily.

In specific embodiments, the maintenance dosing includes administering 600±100 mg per day nefazodone hydrochloride, in divided doses, twice daily.

In specific embodiments, the maintenance dosing includes administering 600±50 mg per day nefazodone hydrochloride, in divided doses, twice daily.

In specific embodiments, the maintenance dosing includes administering 600±25 mg per day nefazodone hydrochloride, in divided doses, twice daily.

In specific embodiments, the maintenance dosing includes administering 600±15 mg per day nefazodone hydrochloride, in divided doses, twice daily.

In specific embodiments, the maintenance dosing includes administering 600±10 mg per day nefazodone hydrochloride, in divided doses, twice daily.

In specific embodiments, the maintenance dosing includes administering 600±5 mg per day nefazodone hydrochloride, in divided doses, twice daily.

In specific embodiments, the maintenance dosing includes administering 600 mg per day nefazodone hydrochloride, in divided doses, twice daily.

In specific embodiments, the maintenance dosing includes administering 400-600 mg per day nefazodone hydrochloride, in divided doses, twice daily.

### Oral Unit Dosage Form

In specific embodiments, the oral unit dosage form contains one or more pharmaceutically acceptable excipients and: (a) 0.1875±0.065 mg risperidone and 25±5 mg nefazodone hydrochloride; (b) 0.55±0.3 mg risperidone and 125±75 mg nefazodone hydrochloride; or (c) 0.875±0.125 mg risperidone and 250±mg nefazodone hydrochloride.

In more specific embodiments, the 0.1875±0.065 mg risperidone in (a) is 0.125 mg risperidone.

In more specific embodiments, the 0.1875±0.065 mg risperidone in (a) is 0.25 mg risperidone.

In more specific embodiments, the 25±5 mg nefazodone hydrochloride in (a) is 25 mg nefazodone hydrochloride.

In more specific embodiments, the 0.55±0.3 mg risperidone in (a) is 0.375 mg risperidone.

In more specific embodiments, the 0.55±0.3 mg risperidone in (a) is 0.5 mg risperidone.

In more specific embodiments, the 0.55±0.3 mg risperidone in (a) is 0.625 mg risperidone.

In more specific embodiments, the 0.55±0.3 mg risperidone in (b) is 0.75 mg risperidone.

In more specific embodiments, the 125±75 mg nefazodone hydrochloride in (b) is 75 mg nefazodone hydrochloride.

In more specific embodiments, the 125±75 mg nefazodone hydrochloride in (b) is 125 mg nefazodone hydrochloride.

In more specific embodiments, the 125±75 mg nefazodone hydrochloride in (b) is 175 mg nefazodone hydrochloride.

In more specific embodiments, the 125±75 mg nefazodone hydrochloride in (b) is 200 mg nefazodone hydrochloride.

In more specific embodiments, the 0.875±0.125 mg risperidone in (c) is 0.75 mg risperidone.

In more specific embodiments, the 250±mg nefazodone hydrochloride in (c) is 200 mg nefazodone hydrochloride.

### Therapeutic Package

In specific embodiments, the therapeutic package includes: (a) a finished pharmaceutical container with printed indicia located thereon; and (b) multiple oral unit dosages contained within the finished pharmaceutical container; wherein, the oral unit dosages include: (i) multiple oral unit dosages, each independently including 0.1875±0.065 mg risperidone and 25±5 mg nefazodone hydrochloride; (ii) multiple oral unit dosages, each independently including 0.55±0.3 mg risperidone and 125±75 mg nefazodone hydrochloride; and/or (iii) multiple oral unit dosages, each independently including 0.875±0.125 mg risperidone and 250±mg nefazodone hydrochloride.

In more specific embodiments, the finished pharmaceutical container includes a plastic pill bottle with closure cap.

In more specific embodiments, the finished pharmaceutical container includes a blister pack.

In more specific embodiments, the printed indicia includes prescribing information.

In more specific embodiments, the 0.1875±0.065 mg risperidone in (i) is 0.125 mg risperidone.

In more specific embodiments, the 0.1875±0.065 mg risperidone in (i) is 0.25 mg risperidone.

In more specific embodiments, the 25±5 mg nefazodone hydrochloride in (i) is 25 mg nefazodone hydrochloride.

In more specific embodiments, the 0.55±0.3 mg risperidone in (i) is 0.375 mg risperidone.

In more specific embodiments, the 0.55±0.3 mg risperidone in (i) is 0.5 mg risperidone.

In more specific embodiments, the 0.55±0.3 mg risperidone in (i) is 0.625 mg risperidone.

In more specific embodiments, the 0.55±0.3 mg risperidone in (ii) is 0.75 mg risperidone.

In more specific embodiments, the 125±75 mg nefazodone hydrochloride in (ii) is 75 mg nefazodone hydrochloride.

In more specific embodiments, the 125±75 mg nefazodone hydrochloride in (ii) is 125 mg nefazodone hydrochloride.

In more specific embodiments, the 125±75 mg nefazodone hydrochloride in (ii) is 175 mg nefazodone hydrochloride.

In more specific embodiments, the 125±75 mg nefazodone hydrochloride in (ii) is 200 mg nefazodone hydrochloride.

In more specific embodiments, the 0.875±0.125 mg risperidone in (iii) is 0.75 mg risperidone.

In more specific embodiments, the 250±mg nefazodone hydrochloride in (iii) is 200 mg nefazodone hydrochloride.

In more specific embodiments, the finished pharmaceutical container, with printed indicia located thereon, contains at least 14 oral unit dosages of 0.1875±0.065 mg risperidone and 25±5 mg nefazodone hydrochloride.

In more specific embodiments, the finished pharmaceutical container, with printed indicia located thereon, contains up to 56 oral unit dosages of 0.1875±0.065 mg risperidone and 25±5 mg nefazodone hydrochloride.

In more specific embodiments, the finished pharmaceutical container, with printed indicia located thereon, contains up to 28 oral unit dosages of 0.1875±0.065 mg risperidone and 25±5 mg nefazodone hydrochloride.

In more specific embodiments, the finished pharmaceutical container, with printed indicia located thereon, contains 14-28 unit dosages of 0.1875±0.065 mg risperidone and 25±5 mg nefazodone hydrochloride.

In more specific embodiments, the finished pharmaceutical container, with printed indicia located thereon, contains at least 28 oral unit dosages of 0.55±0.3 mg risperidone and 125±75 mg nefazodone hydrochloride.

In more specific embodiments, the finished pharmaceutical container, with printed indicia located thereon, contains at least 56 oral unit dosages of 0.55±0.3 mg risperidone and 125±75 mg nefazodone hydrochloride.

In more specific embodiments, the finished pharmaceutical container, with printed indicia located thereon, contains up to 84 oral unit dosages of 0.55±0.3 mg risperidone and 125±75 mg nefazodone hydrochloride.

In more specific embodiments, the finished pharmaceutical container, with printed indicia located thereon, contains 28-84 oral unit dosages of 0.55±0.3 mg risperidone and 125±75 mg nefazodone hydrochloride.

In more specific embodiments, the finished pharmaceutical container, with printed indicia located thereon, contains 42-70 oral unit dosages of 0.55±0.3 mg risperidone and 125±75 mg nefazodone hydrochloride.

In more specific embodiments, the finished pharmaceutical container, with printed indicia located thereon, contains at least 28 oral unit dosages of 1.75±0.3 mg risperidone and 500±125 mg nefazodone hydrochloride.

In more specific embodiments, the finished pharmaceutical container, with printed indicia located thereon, contains at least 56 oral unit dosages of 1.75±0.3 mg risperidone and 500±125 mg nefazodone hydrochloride.

### Marketing & Advertising

In specific embodiments, the pharmaceutical product is marketed, advertised, and/or sold for the treatment, mitigation, and/or reduction of suicidal thoughts and/or suicidal behavior in a person in need thereof.

In specific embodiments, the pharmaceutical product is marketed, advertised, and/or sold for the treatment, mitigation, and/or reduction of depression in a person in need thereof.

In specific embodiments, the pharmaceutical product is marketed, advertised, and/or sold for the treatment, mitigation, and/or reduction of major depressive disorder (MDD) in a person in need thereof.

In specific embodiments, the pharmaceutical product is marketed, advertised, and/or sold to physicians treating people suffering from suicidal thoughts and/or suicidal behavior.

In specific embodiments, the pharmaceutical product is marketed, advertised, and/or sold to prescribers treating people suffering from suicidal thoughts and/or suicidal behavior.

In specific embodiments, the marketing includes the step of including a statement in the labeling for a pharmaceutical product that can treat, mitigate, and/or reduce suicidal thoughts and/or suicidal behavior in a person.

In specific embodiments, the marketing includes the step of including a statement in the labeling for a pharmaceutical product that can treat depression in a person in need thereof.

In specific embodiments, the marketing includes the step of including a statement in the labeling for a pharmaceutical product that can treat major depressive disorder (MDD) in a person in need thereof.

### Unit Dose and Excipients

In specific embodiments, the pharmaceutical product is a unit dose, formulated as an oral dosage form.

In specific embodiments, the pharmaceutical product is a unit dose, formulated as a solid oral dosage form.

In specific embodiments, the pharmaceutical product is a unit dose, formulated as a liquid oral dosage form.

In specific embodiments, the pharmaceutical product is formulated as a syrup, oral solution, or oral suspension.

In specific embodiments, the pharmaceutical product is formulated as an orally disintegrating tablet.

In specific embodiments, the pharmaceutical product is formulated as a lozenge.

In specific embodiments, the pharmaceutical product is formulated as an oral thin film.

In specific embodiments, the pharmaceutical product is formulated as a powder, effervescent powder, or effervescent tablet.

In specific embodiments, the pharmaceutical product is formulated as an oral tablet or an oral capsule.

In specific embodiments, the pharmaceutical product is formulated as an oral tablet.

In specific embodiments, the pharmaceutical product is formulated as an oral tablet that is scored.

In specific embodiments, the pharmaceutical product is formulated as an oral tablet that is not scored.

In specific embodiments, the pharmaceutical product is formulated as an oral capsule.

In specific embodiments, the pharmaceutical product is formulated as an oral tablet or as an oral capsule, and contains one or more diluents, one or more binders, one or more disintegrants, one or more lubricants, one or more glidants, one or more colorants, one or more flavoring agents, one or more sweeteners, one or more surfactants, one or more coating materials, or a combination thereof.

In specific embodiments, the pharmaceutical product is formulated as an oral tablet or as an oral capsule, and contains diluent, binder, disintegrant, lubricant, glidant, colorant, flavoring agent, sweetener, surfactant, coating material, or a combination thereof.

In specific embodiments, the pharmaceutical product is formulated as an oral tablet and contains one or more diluents, one or more disintegrants, and one or more lubricants.

### Monitoring and Safety

In specific embodiments, the pharmaceutical product is marketed, with a warning to allow at least 2 weeks between discontinuing an MAO inhibitor and starting the pharmaceutical product (containing nefazodone).

In specific embodiments, the pharmaceutical product is marketed, with a warning to allow at least 1 week between discontinuing the pharmaceutical product (containing nefazodone) and starting an MAO inhibitor.

In specific embodiments, the pharmaceutical product is marketed, with a warning to allow at least 1 week between discontinuing fluoxetine and initiating the pharmaceutical product (containing nefazodone).

In specific embodiments, the pharmaceutical product is marketed, with a warning to monitor periodically for need for continued therapy.

In specific embodiments, the pharmaceutical product is marketed, with a warning that nefazodone carries a risk of severe, life-threatening hepatic failure.

In specific embodiments, the pharmaceutical product is marketed, with a warning that the patient be monitored by a medical professional, especially during the initial weeks of treatment and any dose adjustments.

In specific embodiments, the pharmaceutical product is marketed, with a warning of discontinuation risks, that abruptly stopping administration of the pharmaceutical product can lead to increased risk of suicidal thoughts and behaviors.

In specific embodiments, the pharmaceutical product is administered to a subject, and the subject is subsequently monitored by a medical professional.

In specific embodiments, the pharmaceutical product is administered to a subject, and the subject is subsequently monitored by a medical professional which includes careful observation for emerging suicidal thoughts or behaviors.

In specific embodiments, the pharmaceutical product is administered to a subject, and during treatment the subject is subsequently monitored by a medical professional.

In specific embodiments, the pharmaceutical product is administered to a subject, and the subject is subsequently monitored by a medical professional during the initial 1-2 weeks of treatment.

In specific embodiments, the pharmaceutical product is administered to a subject, and the subject is subsequently monitored by a medical professional during the treatment.

In specific embodiments, the pharmaceutical product is administered to a subject, and the subject is subsequently monitored by a medical professional during any dose adjustments.

### ENUMERATED EMBODIMENTS

Specific enumerated embodiments <1> to <XX> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Embodiment 1

A method of treating a person suffering from a psychiatric disorder, the method comprising orally administering to the person a unit dose comprising nefazodone hydrochloride, risperidone, and one or more pharmaceutical excipients, the administration comprises a dosing regimen comprising:
an initial dosing comprising administering a first unit dose comprising nefazodone hydrochloride and risperidone,
a dosing escalation comprising administering a second unit dose comprising nefazodone hydrochloride and risperidone, and
a maintenance dosing comprising administering a third unit dose comprising nefazodone hydrochloride and risperidone;
   wherein,
the initial dosing comprises administering a daily dose comprising 50±25 mg nefazodone hydrochloride and 0.375±0.125 mg risperidone;
the initial dosing is carried out for at least 1 week;
the dosing escalation comprises increasing the nefazodone hydrochloride in increments of 100±50 mg per day, until the maintenance dose is achieved;
the dosing escalation comprises increasing the risperidone in increments of 0.375±0.125 mg per day, until the maintenance dose is achieved;
the dosing escalation occurs at intervals of once per at least 1 week;
the dosing escalation occurs over a period of time of at least 2 weeks;
the maintenance dosing comprises administering a daily dose of 500±125 mg nefazodone hydrochloride and 1.75±0.5 mg risperidone; and
each dosing is carried out twice daily (BID) with a solid oral dosage.

### Embodiment 2

The method of embodiment <1>, wherein the initial dosing comprises administering a daily dose of 50 mg nefazodone hydrochloride and 0.25 mg risperidone, twice daily (BID) with a solid oral dosage.

### Embodiment 3

The method of embodiment <1>, wherein the initial dosing comprises administering a daily dose of 50 mg nefazodone hydrochloride and 0.5 mg risperidone, twice daily (BID) with a solid oral dosage.

### Embodiment 4

The method of any one of embodiments <1> to <3>, wherein the initial dosing is carried out for 2 weeks.

### Embodiment 5

The method of any one of embodiments <1> to <4>, wherein the dosing escalation comprises increasing the nefazodone hydrochloride in increments of 100 mg per day, until the maintenance dose is achieved.

### Embodiment 6

The method of any one of embodiments <1> to <5>, wherein the dosing escalation comprises increasing the risperidone in increments of 0.5 mg per day, until the maintenance dose is achieved.

### Embodiment 7

The method of any one of embodiments <1> to <6>, wherein the dosing escalation occurs at an interval of once per week.

### Embodiment 8

The method of any one of embodiments <1> to <7>, wherein the dosing escalation occurs over a period of time of 2-5 weeks.

### Embodiment 9

The method of any one of embodiments <1> to <8>, wherein the maintenance dosing comprises administering a daily dose of 400-600 mg nefazodone hydrochloride and 1.5-2 mg risperidone, twice daily (BID) with a solid oral dosage.

### Embodiment 10

The method of any one of embodiments <1> to <9>, wherein the person is monitored at least once per week by a medical professional for observation of emerging suicidal thoughts or behaviors.

### Embodiment 11

A method of treating a person suffering from a psychiatric disorder, the method comprising orally administering to the person a unit dose comprising nefazodone hydrochloride, risperidone, and one or more pharmaceutical excipients, the administration comprises a dosing regimen comprising:
an initial dosing comprising administering a first unit dose comprising nefazodone hydrochloride and risperidone,
a dosing escalation comprising administering a second unit dose comprising nefazodone hydrochloride and risperidone, and
a maintenance dosing comprising administering a third unit dose comprising nefazodone hydrochloride and risperidone;
   wherein,
the initial dosing comprises administering a daily dose of 50 mg nefazodone hydrochloride and 0.375±0.125 mg risperidone;
the initial dosing is carried out for 2 weeks;
the dosing escalation comprises increasing the nefazodone hydrochloride in increments of 100 mg per day, until the maintenance dose is achieved;
the dosing escalation comprises increasing the risperidone in increments of 0.5 mg per day, until the maintenance dose is achieved;
the dosing escalation occurs at intervals of once per at least 1 week;
the dosing escalation occurs over a period of time of 3-5 weeks;
the maintenance dosing comprises administering a daily dose of 400 mg nefazodone hydrochloride and 1.5 mg risperidone;
each dosing is carried out twice daily (BID) in a solid unit dose; and
the person is monitored at least once per week by a medical professional for observation of emerging suicidal thoughts or behaviors.

### Embodiment 12

A method of treating a person suffering from a psychiatric disorder, the method comprising orally administering to the person a unit dose comprising nefazodone hydrochloride, risperidone, and one or more pharmaceutical excipients, the administration comprises a dosing regimen comprising:
a) an initial daily dose of (i) 0.25 mg risperidone and 50 mg nefazodone hydrochloride, or (ii) 0.50 mg risperidone and 50 mg nefazodone hydrochloride, administered in divided doses twice daily, in a solid oral dosage form, for a period of time of up to 2 weeks;
   i. wherein the daily dose of 0.25 mg risperidone and 50 mg nefazodone hydrochloride is administered BID as a solid oral dosage form comprising 0.125 mg risperidone and 25 mg nefazodone hydrochloride;
   ii. wherein the daily dose of 0.50 mg risperidone and 50 mg nefazodone hydrochloride is administered BID as a solid oral dosage form comprising 0.25 mg risperidone and 25 mg nefazodone hydrochloride;
b) titrated daily doses of (i) 0.75 mg risperidone and 150 mg nefazodone hydrochloride, 1.25 mg risperidone and 250 mg nefazodone hydrochloride, 1.5 mg risperidone and 350 mg nefazodone hydrochloride, and 1.5 mg risperidone and 400 mg nefazodone hydrochloride, or (ii) 1.0 mg risperidone and 150 mg nefazodone hydrochloride, 1.5 mg risperidone and 250 mg nefazodone hydrochloride, 1.5 mg risperidone and 350 mg nefazodone hydrochloride, and 1.5 mg risperidone and 400 mg nefazodone hydrochloride, administered in divided doses twice daily, in a solid oral dosage form, for a period of time of 2-5 weeks;
   i. wherein the daily dose of 0.75 mg risperidone and 150 mg nefazodone hydrochloride is administered BID as a solid oral dosage form comprising 0.375 mg risperidone and 75 mg nefazodone hydrochloride;
   ii. wherein the daily dose of 1.25 mg risperidone and 250 mg nefazodone hydrochloride is administered BID as a solid oral dosage form comprising 0.625 mg risperidone and 125 mg nefazodone hydrochloride;
   iii. wherein the daily dose of 1.5 mg risperidone and 350 mg nefazodone hydrochloride is administered BID as a solid oral dosage form comprising 0.75 mg risperidone and 175 mg nefazodone hydrochloride;
   iv. wherein the daily dose of 1.5 mg risperidone and 400 mg nefazodone hydrochloride is administered BID as a solid oral dosage form comprising 0.75 mg risperidone and 200 mg nefazodone hydrochloride;
   v. wherein the daily dose of 1.0 mg risperidone and 150 mg nefazodone hydrochloride is administered BID as a solid oral dosage form comprising 0.5 mg risperidone and 75 mg nefazodone hydrochloride;
   vi. wherein the daily dose of 1.5 mg risperidone and 250 mg nefazodone hydrochloride is administered BID as a solid oral dosage form comprising 0.75 mg risperidone and 125 mg nefazodone hydrochloride;
   vii. wherein the daily dose of 1.5 mg risperidone and 350 mg nefazodone hydrochloride is administered BID as a solid oral dosage form comprising 0.75 mg risperidone and 175 mg nefazodone hydrochloride;
   viii. wherein the daily dose of 1.5 mg risperidone and 400 mg nefazodone hydrochloride is administered BID as a solid oral dosage form comprising 0.75 mg risperidone and 200 mg nefazodone hydrochloride;
c) maintenance daily dose of (i) 1.5-2 mg risperidone and 400-600 mg nefazodone hydrochloride; and
d) monitoring the person at least once per week by a medical professional for observation of emerging suicidal thoughts or behaviors.

### Embodiment 13

The method of any one of embodiments <1> to <12>, which effectively reduces symptoms associated with the psychiatric disorder.

### Embodiment 14

The method of any one of embodiments <1> to <13>, wherein the psychiatric disorder comprises at least one of suicidal behavior, suicidal ideation, and major depressive disorder (MDD).

### Embodiment 15

The method of any one of embodiments <1> to <14>, which effectively reduces the incidence of suicidal ideation and suicidal behavior in a person suffering from a major depressive episode.

### Embodiment 16

The method of any one of embodiments <1> to <15>, which effectively reduces the incidence of suicidal behavior in a person suffering from a major depressive episode.

### Embodiment 17

The method of any one of embodiments <1> to <16>, which effectively reduces the incidence of suicidal ideation in a person suffering from a major depressive episode.

### Embodiment 18

The method of any one of embodiments <1> to <17>, which effectively reduces the severity of depressive symptoms in the person.

### Embodiment 19

The method of any one of embodiments <1> to <18>, wherein the person is afflicted with treatment-refractory depression.

### Embodiment 20

The method of any one of embodiments <1> to <19>, wherein the unit dose is an oral liquid.

### Embodiment 21

The method of any one of embodiments <1> to <20>, wherein the unit dose is an oral solution, an oral suspension, an oral powder, or oral granules.

### Embodiment 22

The method of any one of embodiments <1> to <21>, wherein the unit dose is an oral solid.

### Embodiment 23

The method of any one of embodiments <1> to <22>, wherein the unit dose is an oral tablet, an oral capsule, or an oral soluble film.

### Embodiment 24

The method of any one of embodiments <1> to <23>, wherein the unit dose is orally administered to the person twice a day (BID).

### Embodiment 25

The method of any one of embodiments <1> to <24>, wherein the person suffering from a psychiatric disorder having a Global Impression of Severity of Suicidality-revised (CGI-SS-r) score of ≥1.

### Embodiment 26

The method of any one of embodiments <1> to <25>, wherein the person was previously prescribed an antipsychotic drug.

### Embodiment 27

The method of any one of embodiments <1> to <26>, wherein the person was previously prescribed an antidepressant drug.

### Embodiment 28

The method of any one of embodiments <1> to <27>, wherein the person is afflicted with treatment-refractory depression.

### Embodiment 29

The method of any one of embodiments <1> to <28>, wherein
the method reduces the incidence of suicidal ideation in a person suffering from a major depressive episode;
the method reduces the incidence of suicidal behavior in a person suffering from a major depressive episode; and
the method reduces the severity of depressive symptoms in the person.

### Embodiment 30

An oral unit dosage form comprising one or more pharmaceutically acceptable excipients and:
(a) 0.1875±0.065 mg risperidone and 25±5 mg nefazodone hydrochloride;
(b) 0.55±0.3 mg risperidone and 125±75 mg nefazodone hydrochloride; or
(c) 0.875±0.125 mg risperidone and 250±mg nefazodone hydrochloride.

### Embodiment 31

A therapeutic package comprising:
(a) finished pharmaceutical container with printed indicial located thereon; and
(b) multiple oral unit dosages contained within the finished pharmaceutical container;
   wherein,
the oral unit dosages comprise:
   (i) multiple oral unit dosages, each independently comprising 0.1875±0.065 mg risperidone and 25±5 mg nefazodone hydrochloride;
   (ii) multiple oral unit dosages, each independently comprising 0.55±0.3 mg risperidone and 125±75 mg nefazodone hydrochloride; and/or
   (iii) multiple oral unit dosages, each independently comprising 0.875±0.125 mg risperidone and 250±mg nefazodone hydrochloride.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

### EXAMPLES

The invention will be illustrated by the following non-limiting examples.

### Formulation Examples

### Example 1A: Components and Composition

### 200 mg Nefazodone Hydrochloride; 0.5 mg Risperidone; Oral Tablet

| % w/w | Ingredient | Function | mg/tablet |
|---|---|---|---|
| 37.00 | Nefazodone | Active | 200.00 |
| 0.09 | Risperidone | Active | 0.50 |
| 37.00 | Microcrystalline Cellulose | Filler/Diluent | 200.00 |
| 23.13 | Lactose Monohydrate | Filler/Diluent | 125.00 |
| | Pharmatose DCL 15) | | |
| 0.93 | Croscarmellose Sodium | Disintegrant | 5.00 |
| | (Primellose) | | |
| 0.93 | Sodium Stearyl Fumarate | Lubricant | 5.00 |
| 0.93 | Colloidal Silicon Dioxide | Lubricant | 5.00 |

| | | |
|---|---|---|
| 100.00 | Total | 540.50 |

### Example 1B: Manufacturing Process and Controls

### 200 mg Nefazodone Hydrochloride; 0.5 mg Risperidone; Oral Tablet

1. Pass all the materials through Sieve 60 mesh equivalent.
2. Charge Risperidone and Microcrystalline Cellulose #1 into V-Blender and mix the material for 10 minutes.
3. Charge Microcrystalline Cellulose #2 into V-blender and mix the material for 10 minutes.
4. Charge Microcrystalline Cellulose #3 into V-blender and mix the material for 10 minutes.
5. Charge Microcrystalline Cellulose #4 into V-blender and mix the material for 10 minutes.
6. Charge Microcrystalline Cellulose #5 into V-blender and mix the material for 10 minutes.
7. Charge Microcrystalline Cellulose #6 into V-blender and mix the material for 10 minutes.
8. Charge Microcrystalline Cellulose #7 into V-blender and mix the material for 10 minutes.
9. Charge Microcrystalline Cellulose #8 into V-blender and mix the material for 10 minutes.
10. Charge Lactose Monohydrate, Croscarmellose Sodium and Sodium Stearyl Fumarate into V-blender and mix the material for 10 minutes.
11. Charge Microcrystalline Cellulose #9 and Nefazodone into V-blender and mix the material for 10 minutes.
12. Pass the blend from step 11 and colloidal silicon dioxide through Sieve 60 mesh equivalent.
13. Charge the material from Step 12 and colloidal silicon dioxide into V-blender and mix the material for 15 minutes.
14. Discharge the product from the Blender. Collect the Blend into labeled, tared containers double-lined with polyethylene bags.
15. Compact the blend from Step 14 using roller compaction process. Pass the compacted blend through 12 #sieve. Collect the Granules into labeled, tared containers double lined with polyethylene bags.
16. Compress the granules into suitable tablets with a target weight of 540.5 mg.

### Example 2A: Oral Dosage Form

The pharmaceutical product described herein can include the following strengths of risperidone and nefazodone, in the specified oral dosage form.

| | | RISPERIDONE | | | | | | | | | |
|---|---|---|---|---|---|---|---|---|---|---|---|
| | | 0.25 ± | 0.3 ± | 0.4 ± | 0.5 ± | 0.75 ± | 1.0 ± | 1.25 ± | 1.5 ± | 1.75 ± | 2.0 ± |
| | | 0.1 mg | 0.1 mg | 0.1 mg | 0.25 mg | 0.25 mg | 0.25 mg | 0.25 mg | 0.25 mg | 0.25 mg | 0.25 mg |
| NEFAZODONE | 50 ± | oral | oral | oral | oral | oral | oral | oral | oral | oral | oral |
| | 25 mg | solid | solid | solid | solid | solid | solid | solid | solid | solid | solid |
| | 75 ± | oral | oral | oral | oral | oral | oral | oral | oral | oral | oral |
| | 25 mg | solid | solid | solid | solid | solid | solid | solid | solid | solid | solid |
| | 100 ± | oral | oral | oral | oral | oral | oral | oral | oral | oral | oral |
| | 25 mg | solid | solid | solid | solid | solid | solid | solid | solid | solid | solid |
| | 125 ± | oral | oral | oral | oral | oral | oral | oral | oral | oral | oral |
| | 25 mg | solid | solid | solid | solid | solid | solid | solid | solid | solid | solid |
| | 150 ± | oral | oral | oral | oral | oral | oral | oral | oral | oral | oral |
| | 25 mg | solid | solid | solid | solid | solid | solid | solid | solid | solid | solid |
| | 175 ± | oral | oral | oral | oral | oral | oral | oral | oral | oral | oral |
| | 25 mg | solid | solid | solid | solid | solid | solid | solid | solid | solid | solid |
| | 200 ± | oral | oral | oral | oral | oral | oral | oral | oral | oral | oral |
| | 25 mg | solid | solid | solid | solid | solid | solid | solid | solid | solid | solid |
| | 225 ± | oral | oral | oral | oral | oral | oral | oral | oral | oral | oral |
| | 25 mg | solid | solid | solid | solid | solid | solid | solid | solid | solid | solid |
| | 250 ± | oral | oral | oral | oral | oral | oral | oral | oral | oral | oral |
| | 25 mg | solid | solid | solid | solid | solid | solid | solid | solid | solid | solid |
| | 275 ± | oral | oral | oral | oral | oral | oral | oral | oral | oral | oral |
| | 25 mg | solid | solid | solid | solid | solid | solid | solid | solid | solid | solid |
| | 300 ± | oral | oral | oral | oral | oral | oral | oral | oral | oral | oral |
| | 25 mg | solid | solid | solid | solid | solid | solid | solid | solid | solid | solid |
| | 325 ± | oral | oral | oral | oral | oral | oral | oral | oral | oral | oral |
| | 25 mg | solid | solid | solid | solid | solid | solid | solid | solid | solid | solid |
| | 350 ± | oral | oral | oral | oral | oral | oral | oral | oral | oral | oral |
| | 25 mg | solid | solid | solid | solid | solid | solid | solid | solid | solid | solid |
| | 375 ± | oral | oral | oral | oral | oral | oral | oral | oral | oral | oral |
| | 25 mg | solid | solid | solid | solid | solid | solid | solid | solid | solid | solid |
| | 400 ± | oral | oral | oral | oral | oral | oral | oral | oral | oral | oral |
| | 25 mg | solid | solid | solid | solid | solid | solid | solid | solid | solid | solid |

### Example 2B: Dosing

The pharmaceutical product described herein can be administered in the following dosages, with the following dosing frequency.

| | | RISPERIDONE | | | | | | | | | |
|---|---|---|---|---|---|---|---|---|---|---|---|
| | | 0.25 ± | 0.3 ± | 0.4 ± | 0.5 ± | 0.75 ± | 1.0 ± | 1.25 ± | 1.5 ± | 1.75 ± | 2.0 ± |
| | | 0.1 mg | 0.1 mg | 0.1 mg | 0.25 mg | 0.25 mg | 0.25 mg | 0.25 mg | 0.25 mg | 0.25 mg | 0.25 mg |
| NEFAZODONE | 50 ± | BID | BID | BID | BID | BID | BID | BID | BID | BID | BID |
| | 25 mg | | | | | | | | | | |
| | 75 ± | BID | BID | BID | BID | BID | BID | BID | BID | BID | BID |
| | 25 mg | | | | | | | | | | |
| | 100 ± | BID | BID | BID | BID | BID | BID | BID | BID | BID | BID |
| | 25 mg | | | | | | | | | | |
| | 125 ± | BID | BID | BID | BID | BID | BID | BID | BID | BID | BID |
| | 25 mg | | | | | | | | | | |
| | 150 ± | BID | BID | BID | BID | BID | BID | BID | BID | BID | BID |
| | 25 mg | | | | | | | | | | |
| | 175 ± | BID | BID | BID | BID | BID | BID | BID | BID | BID | BID |
| | 25 mg | | | | | | | | | | |
| | 200 ± | BID | BID | BID | BID | BID | BID | BID | BID | BID | BID |
| | 25 mg | | | | | | | | | | |
| | 225 ± | BID | BID | BID | BID | BID | BID | BID | BID | BID | BID |
| | 25 mg | | | | | | | | | | |
| | 250 ± | BID | BID | BID | BID | BID | BID | BID | BID | BID | BID |
| | 25 mg | | | | | | | | | | |
| | 275 ± | BID | BID | BID | BID | BID | BID | BID | BID | BID | BID |
| | 25 mg | | | | | | | | | | |
| | 300 ± | BID | BID | BID | BID | BID | BID | BID | BID | BID | BID |
| | 25 mg | | | | | | | | | | |
| | 325 ± | BID | BID | BID | BID | BID | BID | BID | BID | BID | BID |
| | 25 mg | | | | | | | | | | |
| | 350 ± | BID | BID | BID | BID | BID | BID | BID | BID | BID | BID |
| | 25 mg | | | | | | | | | | |
| | 375 ± | BID | BID | BID | BID | BID | BID | BID | BID | BID | BID |
| | 25 mg | | | | | | | | | | |
| | 400 ± | BID | BID | BID | BID | BID | BID | BID | BID | BID | BID |
| | 25 mg | | | | | | | | | | |
| | 425 ± | BID | BID | BID | BID | BID | BID | BID | BID | BID | BID |
| | 25 mg | | | | | | | | | | |
| | 450 ± | BID | BID | BID | BID | BID | BID | BID | BID | BID | BID |
| | 25 mg | | | | | | | | | | |
| | 500 ± | BID | BID | BID | BID | BID | BID | BID | BID | BID | BID |
| | 25 mg | | | | | | | | | | |
| | 525 ± | BID | BID | BID | BID | BID | BID | BID | BID | BID | BID |
| | 25 mg | | | | | | | | | | |
| | 550 ± | BID | BID | BID | BID | BID | BID | BID | BID | BID | BID |
| | 25 mg | | | | | | | | | | |
| | 575 ± | BID | BID | BID | BID | BID | BID | BID | BID | BID | BID |
| | 25 mg | | | | | | | | | | |
| | 600 ± | BID | BID | BID | BID | BID | BID | BID | BID | BID | BID |
| | 25 mg | | | | | | | | | | |

### Example 2C: Dosing Regimen

| | Oral Unit Dosage Form | |
|---|---|---|
| Dosing Parameters: | Risperidone | Nefazodone hydrochloride |
| Initial dosing (strength) | 0.25-0.5 mg per day | 50 mg per day |
| Initial dosing duration (period of | 1-2 weeks | 1-2 weeks |
| time) | | |
| Dosage escalation (amount) | 0.5 mg per day | 100 mg per day |
| Dosage escalation (interval between | weekly (1 week or greater) | weekly (1 week or greater) |
| adjustments) | | |
| Dosage escalation (duration of | 1-4 weeks | 1-4 weeks |
| escalation period) | | |
| Maintenance dosing (strength) | 1.5 mg per day | 400 mg per day |
| Maintenance dosing (period of time) | ongoing | ongoing |

| | |
|---|---|
| Dosing frequency | BID |

### Example 2D: Unit Dosages for Daily Administration

| | Daily Administration (BID) | | | | Oral Unit Dosage Form | | | |
|---|---|---|---|---|---|---|---|---|
| | | | Nefazodone | | | | Nefazodone | |
| | Risperidone | | hydrochloride | | Risperidone | | hydrochloride | |
| Initial Dose | 0.25 | mg | 50 | mg | 0.125 | mg | 25 | mg |
| (Option A) | | | | | | | | |
| Initial Dose | 0.50 | mg | 50 | mg | 0.25 | mg | 25 | mg |
| (Option B) | | | | | | | | |
| Titrated Doses | 0.75 | mg | 150 | mg | 0.375 | mg | 75 | mg |
| (Option A) | 1.25 | mg | 250 | mg | 0.625 | mg | 125 | mg |
| | 1.5 | mg | 350 | mg | 0.75 | mg | 175 | mg |
| Titrated Doses | 1.0 | mg | 150 | mg | 0.5 | mg | 75 | mg |
| (Option B) | 1.5 | mg | 250 | mg | 0.75 | mg | 125 | mg |
| | 1.5 | mg | 350 | mg | 0.75 | mg | 175 | mg |
| Maintenance Dose | 1.5 | mg | 400 | mg | 0.75 | mg | 200 | mg |

### Example 3A: Subject Population

Key inclusion and exclusion criteria for candidate subjects for the medical treatment described herein are provided below.

### Key Inclusion Criteria

Subject meets Diagnostic and Statistical Manual of Mental Disorders (5th edition) (DSM-5) diagnostic criteria for a MDE, based upon clinical assessment and confirmed by the Mini International Psychiatric Interview (MINI).
Subjects has chronic suicidal ideation, confirmed by a score of ≥3 (at least moderate) in the CGI-SS-r for at least 6 weeks (a suicidal ideation for most of the day, for more days than not) prior to screening.
Subject has a MADRS total score of ≥24 at Screening and Baseline
Subject is receiving a therapeutic dose of an antidepressant treatment (ADT) with or without augmentation for at least 8 weeks prior to screening.
Subject can, in the opinion of the investigator, be safely managed as an outpatient for the entire treatment.

### Key Exclusion Criteria

Subject has a current or prior DSM-5 diagnosis of a psychotic disorder, or an MDE with psychotic symptoms.
Subject meets the DSM-5 criteria for substance use disorder (except for nicotine or caffeine) within 6 months before screening.

Criteria used to identify candidate subjects to receive the medical treatment described herein are described below.

### Example 3B: Clinical Global Impression of Severity of Suicidality-Revised (CGI-SS-r)

Considering your total clinical experience with suicidal patients and all information now available to you, how suicidal is the patient at this time?
0. Normal, not at all suicidal
1. Questionably suicidal
2. Mildly suicidal
3. Moderately suicidal
4. Markedly suicidal
5. Severely suicidal
6. Among the most extremely suicidal patients

| SCALE | RATING | GUIDE TO RATING (CGI-SS-r) |
|---|---|---|
| 0 | Normal, not at all suicidal | Not suicidal |
| 1 | Questionably suicidal | Minimal ideations; little if any impulsivity for |
| | | suicide, few risk factors and many protective |
| | | factors; and no impact on function. |
| 2 | Mildly suicidal | Occasional ideations; little if any impulsivity for |
| | | suicide; few risk factors; adequate protective |
| | | factors and no or minimal impact on function. |
| 3 | Moderately suicidal | Intermittent ideations; with possible impulsivity |
| | | for suicide; may or may not have plan or recent |
| | | attempt*; several risk factors; protective factors |
| | | may outweigh risk factors and some impact on |
| | | function. |
| 4 | Markedly suicidal | Regular ideations with intent or potential for |
| | | impulsive actions for suicide; may or may not have |
| | | plan or recent attempt*; multiple risk factors |
| | | outweigh protective factors; and marked impact on |
| | | function. |
| 5 | Severely suicidal | Frequent ideations with intent; well worked out |
| | | suicide plan; may or may not have recent attempt*; |
| | | multiple risk factors outweigh protective factors; |
| | | and major impact on function. |
| 6 | Among the most extremely | Nearly constant suicidal ideations and intent; well |
| | suicidal patients | worked out plan and preparations underway or |
| | | recent attempt*; and severe impact on function. |

*Consider seriousness/lethality of any plan or suicide attempt in overall rating

### Example 3C: FoST

Considering all of the information available to you, what is the patient's frequency of suicidal thinking at this time?
Never
Rarely
Sometimes
Often
Most of the time
All of the time

### Example 4: Clinical Evaluation and Diagnosis of Depression

Patients were clinically evaluated for depression by medical personnel. A diagnosis of depression was arrived at by conducting a thorough clinical interview and assessment of the subject patient. This typically included a discussion of the individual's symptoms, medical and psychiatric history, and any current stressors or life events. They may also have used standardized questionnaires or rating scales to help evaluate the severity of symptoms. In some cases, diagnostic imaging or laboratory tests were used to rule out other possible causes of symptoms. Ultimately, the diagnosis of depression was based on the criteria outlined in the Diagnostic and Statistical Manual of Mental Disorders (DSM-5) or the International Classification of Diseases (ICD-11).

### Example 5: Questions Used to Identify a Suicidal Patient

Questions used to identify a suicidal patient include:
1. “Are you thinking about hurting yourself or attempting suicide?”
2. “Do you have a plan for how you would go about hurting yourself or committing suicide?”
3. “Do you have access to the means (e.g., pills, weapons) to hurt yourself or commit suicide?”
4. “Have you been feeling hopeless or helpless lately?”
5. “Do you feel like you would be better off dead or that the world would be a better place without you?”
6. “Have you been experiencing a significant loss or change in your life recently?”
7. “Are you experiencing overwhelming stress or emotional pain?”
8. “Are you experiencing severe depression or other mental health issues?”

### Example 6: Questionnaires Used to Diagnose Major Depression

Questionnaires used to diagnose major depression, include:
1. Beck Depression Inventory (BDI): A widely used self-report questionnaire that measures the severity of depression symptoms.
2. Hamilton Depression Rating Scale (HDRS): A clinician-administered questionnaire that rates the severity of depression symptoms on a scale of 0-52.
3. Patient Health Questionnaire (PHQ-9): A self-administered questionnaire that assesses the presence and severity of depression symptoms according to the criteria of the DSM.
4. Zung Self-Rating Depression Scale (SDS): A self-report questionnaire that assesses the presence and severity of depression symptoms on a scale of 20-80.
5. Center for Epidemiological Studies Depression Scale (CES-D): A self-report questionnaire that measures the presence and severity of depression symptoms in community samples.
6. Columbia Suicide Severity Rating Scale (C-SSRS): Rating scale designed to provide a prospective, standardized measure of suicidality. The scale allows clinicians and researchers alike to assess the severity and lethality of suicidal behaviors and ideations, and can be used to monitor treatment outcomes and establish suicide risk in a variety of research and clinical settings.

1. A method of treating a person suffering from a psychiatric disorder, the method comprising orally administering to the person a unit dose comprising nefazodone hydrochloride, risperidone, and one or more pharmaceutical excipients, the administration comprises a dosing regimen comprising:
an initial dosing comprising administering a first unit dose comprising nefazodone hydrochloride and risperidone,
a dosing escalation comprising administering a second unit dose comprising nefazodone hydrochloride and risperidone, and
a maintenance dosing comprising administering a third unit dose comprising nefazodone hydrochloride and risperidone;
wherein,
the initial dosing comprises administering a daily dose comprising 50±25 mg nefazodone hydrochloride and 0.375±0.125 mg risperidone;
the initial dosing is carried out for at least 1 week;
the dosing escalation comprises increasing the nefazodone hydrochloride in increments of 100±50 mg per day, until the maintenance dose is achieved;
the dosing escalation comprises increasing the risperidone in increments of 0.375±0.125 mg per day, until the maintenance dose is achieved;
the dosing escalation occurs at intervals of once per at least 1 week;
the dosing escalation occurs over a period of time of at least 2 weeks;
the maintenance dosing comprises administering a daily dose of 500±125 mg nefazodone hydrochloride and 1.75±0.5 mg risperidone; and
each dosing is carried out twice daily (BID) with a solid oral dosage.

2. The method of claim 1, wherein the initial dosing comprises administering a daily dose of 50 mg nefazodone hydrochloride and 0.25 mg risperidone, twice daily (BID) with a solid oral dosage.

3. The method of claim 1, wherein the initial dosing comprises administering a daily dose of 50 mg nefazodone hydrochloride and 0.5 mg risperidone, twice daily (BID) with a solid oral dosage.

4. The method of claim 1, wherein the initial dosing is carried out for 2 weeks.

5. The method of claim 1, wherein the dosing escalation comprises increasing the nefazodone hydrochloride in increments of 100 mg per day, until the maintenance dose is achieved.

6. The method of claim 1, wherein the dosing escalation comprises increasing the risperidone in increments of 0.5 mg per day, until the maintenance dose is achieved.

7. The method of claim 1, wherein the dosing escalation occurs at an interval of once per week.

8. The method of claim 1, wherein the dosing escalation occurs over a period of time of 2-5 weeks.

9. The method of claim 1, wherein the maintenance dosing comprises administering a daily dose of 400-600 mg nefazodone hydrochloride and 1.5-2 mg risperidone, twice daily (BID) with a solid oral dosage.

10. The method of claim 1, wherein the person is monitored at least once per week by a medical professional for observation of emerging suicidal thoughts or behaviors.

11. A method of treating a person suffering from a psychiatric disorder, the method comprising orally administering to the person a unit dose comprising nefazodone hydrochloride, risperidone, and one or more pharmaceutical excipients, the administration comprises a dosing regimen comprising:
an initial dosing comprising administering a first unit dose comprising nefazodone hydrochloride and risperidone,
a dosing escalation comprising administering a second unit dose comprising nefazodone hydrochloride and risperidone, and
a maintenance dosing comprising administering a third unit dose comprising nefazodone hydrochloride and risperidone;
wherein,
the initial dosing comprises administering a daily dose of 50 mg nefazodone hydrochloride and 0.375±0.125 mg risperidone;
the initial dosing is carried out for 2 weeks;
the dosing escalation comprises increasing the nefazodone hydrochloride in increments of 100 mg per day, until the maintenance dose is achieved;
the dosing escalation comprises increasing the risperidone in increments of 0.5 mg per day, until the maintenance dose is achieved;
the dosing escalation occurs at intervals of once per at least 1 week;
the dosing escalation occurs over a period of time of 3-5 weeks;
the maintenance dosing comprises administering a daily dose of 400 mg nefazodone hydrochloride and 1.5 mg risperidone;
each dosing is carried out twice daily (BID) in a solid unit dose; and
the person is monitored at least once per week by a medical professional for observation of emerging suicidal thoughts or behaviors.

12. A method of treating a person suffering from a psychiatric disorder, the method comprising orally administering to the person a unit dose comprising nefazodone hydrochloride, risperidone, and one or more pharmaceutical excipients, the administration comprises a dosing regimen comprising:
a) an initial daily dose of (i) 0.25 mg risperidone and 50 mg nefazodone hydrochloride, or (ii) 0.50 mg risperidone and 50 mg nefazodone hydrochloride, administered in divided doses twice daily, in a solid oral dosage form, for a period of time of up to 2 weeks;
i. wherein the daily dose of 0.25 mg risperidone and 50 mg nefazodone hydrochloride is administered BID as a solid oral dosage form comprising 0.125 mg risperidone and 25 mg nefazodone hydrochloride;
ii. wherein the daily dose of 0.50 mg risperidone and 50 mg nefazodone hydrochloride is administered BID as a solid oral dosage form comprising 0.25 mg risperidone and 25 mg nefazodone hydrochloride;
b) titrated daily doses of (i) 0.75 mg risperidone and 150 mg nefazodone hydrochloride, 1.25 mg risperidone and 250 mg nefazodone hydrochloride, 1.5 mg risperidone and 350 mg nefazodone hydrochloride, and 1.5 mg risperidone and 400 mg nefazodone hydrochloride, or (ii) 1.0 mg risperidone and 150 mg nefazodone hydrochloride, 1.5 mg risperidone and 250 mg nefazodone hydrochloride, 1.5 mg risperidone and 350 mg nefazodone hydrochloride, and 1.5 mg risperidone and 400 mg nefazodone hydrochloride, administered in divided doses twice daily, in a solid oral dosage form, for a period of time of 2-5 weeks;
i. wherein the daily dose of 0.75 mg risperidone and 150 mg nefazodone hydrochloride is administered BID as a solid oral dosage form comprising 0.375 mg risperidone and 75 mg nefazodone hydrochloride;
ii. wherein the daily dose of 1.25 mg risperidone and 250 mg nefazodone hydrochloride is administered BID as a solid oral dosage form comprising 0.625 mg risperidone and 125 mg nefazodone hydrochloride;
iii. wherein the daily dose of 1.5 mg risperidone and 350 mg nefazodone hydrochloride is administered BID as a solid oral dosage form comprising 0.75 mg risperidone and 175 mg nefazodone hydrochloride;
iv. wherein the daily dose of 1.5 mg risperidone and 400 mg nefazodone hydrochloride is administered BID as a solid oral dosage form comprising 0.75 mg risperidone and 200 mg nefazodone hydrochloride;
v. wherein the daily dose of 1.0 mg risperidone and 150 mg nefazodone hydrochloride is administered BID as a solid oral dosage form comprising 0.5 mg risperidone and 75 mg nefazodone hydrochloride;
vi. wherein the daily dose of 1.5 mg risperidone and 250 mg nefazodone hydrochloride is administered BID as a solid oral dosage form comprising 0.75 mg risperidone and 125 mg nefazodone hydrochloride;
vii. wherein the daily dose of 1.5 mg risperidone and 350 mg nefazodone hydrochloride is administered BID as a solid oral dosage form comprising 0.75 mg risperidone and 175 mg nefazodone hydrochloride;
viii. wherein the daily dose of 1.5 mg risperidone and 400 mg nefazodone hydrochloride is administered BID as a solid oral dosage form comprising 0.75 mg risperidone and 200 mg nefazodone hydrochloride;
c) maintenance daily dose of (i) 1.5-2 mg risperidone and 400-600 mg nefazodone hydrochloride; and
d) monitoring the person at least once per week by a medical professional for observation of emerging suicidal thoughts or behaviors.

13. The method of claim 1, which effectively reduces symptoms associated with the psychiatric disorder.

14. The method of claim 1, wherein the psychiatric disorder comprises at least one of suicidal behavior, suicidal ideation, and major depressive disorder (MDD).

15. The method of claim 1, which effectively reduces the incidence of suicidal ideation and suicidal behavior in a person suffering from a major depressive episode.

16. The method of claim 1, which effectively reduces the incidence of suicidal behavior in a person suffering from a major depressive episode.

17. The method of claim 1, which effectively reduces the incidence of suicidal ideation in a person suffering from a major depressive episode.

18. The method of claim 1, which effectively reduces the severity of depressive symptoms in the person.

19. The method of claim 1, wherein the person is afflicted with treatment-refractory depression.

20. The method of claim 1, wherein the unit dose is an oral liquid.

21. The method of claim 1, wherein the unit dose is an oral solution, an oral suspension, an oral powder, or oral granules.

22. The method of claim 1, wherein the unit dose is an oral solid.

23. The method of claim 1, wherein the unit dose is an oral tablet, an oral capsule, or an oral soluble film.

24. The method of claim 1, wherein the unit dose is orally administered to the person twice a day (BID).

25. The method of claim 1, wherein the person suffering from a psychiatric disorder having a Global Impression of Severity of Suicidality-revised (CGI-SS-r) score of ≥1.

26. The method of claim 1, wherein the person was previously prescribed an antipsychotic drug.

27. The method of claim 1, wherein the person was previously prescribed an antidepressant drug.

28. The method of claim 1, wherein the person is afflicted with treatment-refractory depression.

29. The method of claim 1, wherein
the method reduces the incidence of suicidal ideation in a person suffering from a major depressive episode;
the method reduces the incidence of suicidal behavior in a person suffering from a major depressive episode; and
the method reduces the severity of depressive symptoms in the person.

30. An oral unit dosage form comprising one or more pharmaceutically acceptable excipients and:
(a) 0.1875±0.065 mg risperidone and 25±5 mg nefazodone hydrochloride;
(b) 0.55±0.3 mg risperidone and 125±75 mg nefazodone hydrochloride; or
(c) 0.875±0.125 mg risperidone and 250±mg nefazodone hydrochloride.

31. A therapeutic package comprising:
(a) finished pharmaceutical container with printed indicial located thereon; and
(b) multiple oral unit dosages contained within the finished pharmaceutical container;
wherein,
the oral unit dosages comprise:
(i) multiple oral unit dosages, each independently comprising 0.1875±0.065 mg risperidone and 25±5 mg nefazodone hydrochloride;
(ii) multiple oral unit dosages, each independently comprising 0.55±0.3 mg risperidone and 125±75 mg nefazodone hydrochloride; and/or
(iii) multiple oral unit dosages, each independently comprising 0.875±0.125 mg risperidone and 250±mg nefazodone hydrochloride.
